(19) United States
(12) Patent Application Publication
(10) Pub. No.: US 2009/0221687 A1
(43) Pub. Date: Sep. 3, 2009

(54) Tetracyclic Monoamine Reuptake Inhibitors for Treatment of Cns Diseases and Disorders

(76) Inventors: Milan Mesic, Zagreb (HR); Mladen Mercep, Zagreb (HR); Dijana Pesic, Sibenik (HR); Renata Rupcic, Zagreb (HR); Barbara Stanic, Zagreb (HR)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/815,317

(22) PCT Filed: Feb. 1, 2006
(86) PCT No.: PCT/IB06/01480

§ 371 Date: Mar. 30, 2009

Related U.S. Application Data
(60) Provisional application No. 60/649,807, filed on Feb. 2, 2005.

(51) Int. Cl. A61K 31/381; C07D 495/04; A61P 25/00

(52) U.S. Cl. 514/443; 549/42

(57) ABSTRACT

Novel tetracyclic dibenzo(e,h)azulene compounds of formula I; their pharmacologically acceptable derivatives; process and intermediates for their preparation; pharmaceutical compositions containing them and their activity and use in the treatment of central nervous system (CNS) diseases and conditions in humans and animals.

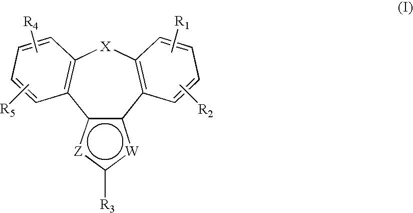

### FIELD OF THE INVENTION

The present invention relates to: (a) novel tetracyclic dibenzo(e,h)azulene compounds; (b) their pharmacologically acceptable derivatives; (c) process and intermediates for their preparation; (d) to pharmaceutical compositions containing them and (e) their activity and use in the treatment of central nervous system (CNS) diseases and conditions in humans and animals.

### TECHNICAL PROBLEM

The present invention addresses the problem of effective treatment (optimally with an improved side effect profile) of diseases, damages and disorders of the central nervous system as well as the treatment of conditions and states that involve various types of pain. The novel tetracyclic dibenzo(e,h)azulene compounds of the invention are triple monoamine neurotransmitter reuptake inhibitors, and thus better suited to achieve global monoamine reuptake inhibition.

### BACKGROUND OF THE INVENTION

The structure of the communication network in the brain consists of neurons that communicate with each other via chemicals recognized as messengers called neurochemicals or neurotransmitters. These neurotransmitters are produced by neurons and they act at sites termed receptors on the cellular membrane of neurons. One group of neurotransmitters, referred to as the monoamine neurotransmitters, includes serotonin, dopamine and norepinephrine.

Monoamine neurotransmitters are released into the synaptic cleft between neurons and they act by stimulation of a postsynaptic receptor. The removal (or inactivation) of monoamine neurotransmitters occurs mainly by a reuptake mechanism into the presynaptic terminals. In various diseases and conditions wherein neurotransmitters are not in balance, enhancement of physiological conditions can occur by inhibiting the reuptake of a particular neurotransmitter. This can then lead to an improvement of the patient's condition.

Selective Serotonin Reuptake Inhibitors (SSRIs) currently provide efficacy in the treatment of major depressive disorder (MDD) and are generally perceived by psychiatrists and primary care physicians as effective, well-tolerated and easily administered. However, SSRIs are also associated with undesirable features, such as high incidence of sexual dysfunction, delayed onset of action and a level of non-responsiveness estimated to be as high as 30% (see M. J. Gitlin, Journal of Clinical Psychiatry, 1994, 55, 406-413 and R. T. Segraves, Journal of Clinical Psychiatry, 1992, 10(2), 4-10). Preclinical and clinical evidence has indicated that the sexual dysfunction associated with SSRI therapy can be reduced through the use of dopamine reuptake inhibitors (DRIs), such as bupropion (see A. K. Ashton, Journal of Clinical Psychiatry, 1998, 59(3), 112-115). Furthermore, the combination of SSRI and DRI may hasten the onset of action and offer relief to refractory patients, possibly through a synergistic mechanism (see R. O. Marshall et al, Journal of Psychopharmacology, 1995, 9(3), 284-286).

Moreover, dual serotonin and norepinephrine reuptake inhibitors (SNRIs) have been tested for chronic pain conditions and have been found effective particularly in fibromyalgia. Although prostaglandin inhibitors have been used for the treatment of acute pain, particularly pain associated with inflammation, their efficacy is limited to milder types of pain and they often display undesirable side effects in the gastrointestinal tract and liver. Narcotics are also used to treat pain, but tolerance develops rapidly and higher doses eventually lead to physical dependence and additional side effects, including respiratory depression. Such therapy is therefore not appropriate or effective for chronic pain conditions.

The combination of triple SSRI, NRI, DRI therapy may be more effective than SSRI or SNRI therapy alone, possibly through an additional synergistic mechanism of a DRI. Additionally, preclinical and clinical evidence has indicated that the sexual dysfunction associated with SSRI therapy can be reduced through the use of DRIs.

Some dibenzo(e,h)azulene compounds, their pharmacologically acceptable salts and solvates, processes and intermediates for their preparation, and their use in the treatment of inflammatory diseases and conditions in humans and animals are disclosed in International publications: WO 01/87890; WO 03/097649; WO 03/097648; WO 03/099823; WO 03/099827; WO 03/084964 and WO 03/084961.

International Publication No. WO 04/078763 discloses further tetracyclic dibenzoazulene compounds having an aminoalkoxy chain connected to the benzene rings, their pharmacologically acceptable salts and solvates, processes and intermediates for their preparation, and their use in the treatment of inflammatory diseases and conditions in humans and animals.

### SUMMARY OF THE INVENTION

New tetracyclic dibenzo(e,h)azulene compounds having both halogen and aminoalkyl or aminoheterocyclic moiety connected to the same benzene ring, represent the subject of the present invention.

Thus, the present invention is directed to compounds represented by Formula I

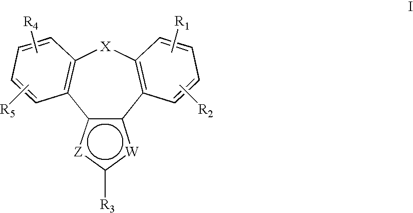

wherein,
X is selected from —CH₂—, —O—, —S—, or NR⁶, wherein R⁶ has meaning of hydrogen, C₁-C₄ alkyl, C₇-C₁₀ arylalkyl, C₂-C₅ alkanoyl, C₇-C₁₀ aryloyl, or C₂-C₇ alkyloxycarbonyl;
W and Z are independently selected from oxygen, sulfur, an aromatic CH, or NR⁷ wherein R⁷ has meaning of hydrogen, C₁-C₄ alkyl, C₇-C₁₀ arylalkyl, C₂-C₅ alkanoyl, C₇-C₁₀ aryloyl, or C₂-C₇ alkyloxycarbonyl; with a proviso that W and Z cannot simultaneously be oxygen, sulfur, or an aromatic CH;
R¹ denotes a substituent represented by Formula II:

Q₁-(CH₂)n-Q₂-(CH₂)p-A II
wherein Q₁ and Q₂ are independently selected from oxygen, sulfur or a group selected from:

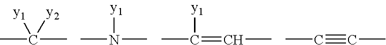

wherein Y₁ is selected from hydrogen; C₁-C₄ alkyl which is unsubstituted or is substituted by 1 to 3 substituents selected from the group consisting of halogen (preferably fluorine or chlorine), hydroxy, C₁-C₄ alkoxy (preferably methoxy or ethoxy), C₁-C₄ alkoxycarbonyl (preferably methoxycarbonyl or ethoxycarbonyl), thiol, C₁-C₄ alkylthio (preferably methylthio or ethylthio), amino, N—(C₁-C₄) alkylamino (preferably N-methylamino or N ethylamino), N,N-di(C₁-C₄ alkyl)-amino preferably dimethylamino or diethylamino), sulfonyl, C₁-C₄ alkylsulfonyl (preferably methylsulfonyl or ethylsulfonyl), sulfinyl and C₁-C₄ alkylsulfinyl (preferably methylsulfinyl); aryl which is unsubstituted or is substituted by one or two substituents selected from the group consisting of halogen (preferably chlorine or fluorine), C₁-C₄ alkyl (preferably methyl, ethyl or isopropyl), cyano, nitro, hydroxy, C₁-C₄ alkoxy (preferably methoxy or ethoxy), thiol, C₁-C₄ alkylthio (preferably methylthio or ethylthio), amino, N—(C₁-C₄) alkylamino (preferably N-methylamino or N-ethylamino), N,N-di(C₁-C₄ alkyl)-amino (preferably N,N-dimethylamino or N,N-diethylamino), sulfonyl, C₁-C₄ alkylsulfonyl (preferably methylsulfonyl or ethylsulfonyl), sulfinyl and C₁-C₄ alkylsulfinyl (preferably methylsulfinyl); hydroxy; C₁-C₄ alkoxy; C₁-C₄ alkanoyl; sulfonyl; C₁-C₄ alkylsulfonyl; sulfinyl; C₁-C₄ alkylsulfinyl; and
Y₂ is selected from hydrogen; halogen; C₁-C₄ alkyl which is unsubstituted or is substituted by 1 to 3 substituents selected from the group consisting of halogen (preferably fluorine or chlorine), hydroxy, C₁-C₄ alkoxy preferably methoxy or ethoxy), C₁-C₄ alkoxycarbonyl (preferably methoxycarbonyl or ethoxycarbonyl), thiol, C₁-C₄ alkylthio (preferably methylthio or ethylthio), amino, N—(C₁-C₄) alkylamino (preferably N-methylamino or N ethylamino), N,N-di(C₁-C₄ alkyl)-amino (preferably dimethylamino or diethylamino), sulfonyl, C₁-C₄ alkylsulfonyl preferably methylsulfonyl or ethylsulfonyl), sulfinyl and C₁-C₄ alkylsulfinyl (preferably methylsulfinyl); aryl which is unsubstituted or is substituted by one or two substituents selected from the group consisting of halogen (preferably chlorine or fluorine), C₁-C₄ alkyl (preferably methyl, ethyl or isopropyl), cyano, nitro, hydroxy, C₁-C₄ alkoxy preferably methoxy or ethoxy), thiol, C₁-C₄ alkylthio (preferably methylthio or ethylthio), amino, N—(C₁-C₄)alkylamino (preferably N-methylamino or N-ethylamino), N,N-di(C₁-C₄ alkyl)-amino (preferably N,N-dimethylamino or N,N-diethylamino), sulfonyl, C₁-C₄ alkylsulfonyl (preferably methylsulfonyl or ethylsulfonyl), sulfinyl and C₁-C₄ alkylsulfinyl (preferably methylsulfinyl); hydroxy; C₁-C₄ alkoxy; C₁-C₄ alkanoyl; thiol; C₁-C₄ alkylthio; sulfonyl; C₁-C₄ alkylsulfonyl; sulfinyl; C₁-C₄ alkylsulfinyl; cyano; nitro;
with a proviso that at least one of substituents Y₁ and Y₂ when attached to carbon atom has meaning of hydrogen.
A is selected from amino, N—(C₁-C₇ allyl)amino, N,N-di(C₁-C₇ alkyl)amino, aryl which is unsubstituted or is substituted by one or two substituents selected from the group consisting of halogen (preferably chlorine or fluorine), C₁-C₄ alkyl (preferably methyl, ethyl or isopropyl), cyano, nitro, hydroxy, C₁-C₄ alkoxy (preferably methoxy or ethoxy), thiol, C₁-C₄ alkylthio (preferably methylthio or ethylthio), amino, N—(C₁-C₄)alkylamino (preferably N-methylamino or N-ethylamino), N,N-di(C₁-C₄-allyl)-amino (preferably N,N-dimethylamino or N,N-diethylamino), sulfonyl, C₁-C₄ alkylsulfonyl (preferably methylsulfonyl or ethylsulfonyl), sulfinyl and C₁-C₄ alkylsulfinyl (preferably methylsulfinyl); heterocyclic or heteroaryl group selected from the group consisting of morpholine-4-yl, piperidine-1-yl, pyrrolidine-1-yl, imidazole-1-yl and piperazine-1-yl, or represented by the structure of Formula III:

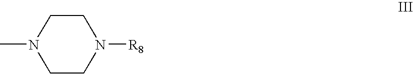

wherein R⁸ is selected from hydrogen; C₁-C₇ alkyl which is unsubstituted or is substituted by 1 to 3 substituents selected from the group consisting of halogen (preferably fluorine or chlorine), hydroxy, C₁-C₄ alkoxy (preferably methoxy or ethoxy), C₁-C₄ alkoxycarbonyl (preferably methoxycarbonyl or ethoxycarbonyl), thiol, C₁-C₄ alkylthio (preferably methylthio or ethylthio), amino, N—(C₁-C₄) alkylamino (preferably N-methylamino or N ethylamino), N,N-di(C₁-C₄ alkyl)-amino (preferably dimethylamino or diethylamino), sulfonyl, C₁-C₄ alkylsulfonyl (preferably methylsulfonyl or ethylsulfonyl), sulfinyl and C₁-C₄ alkylsulfinyl (preferably methylsulfinyl); C₂-C₇ alkenyl which is unsubstituted or is substituted by 1 to 3 halogen (preferably chlorine or fluorine); C₂-C₇ alkynyl, aryl which is unsubstituted or is substituted by one or two substituents selected from the group consisting of halogen (preferably chlorine or fluorine), C₁-C₄ alkyl (preferably methyl, ethyl or isopropyl), cyano, nitro, hydroxy, C₁-C₄ alkoxy (preferably methoxy or ethoxy), C₁-C₄ alkoxycarbonyl (preferably methoxycarbonyl or ethoxycarbonyl), thiol, C₁-C₄ alkylthio (preferably methylthio or ethylthio), amino, N—(C₁-C₄) alkylamino (preferably N-methylamino or N-ethylamino), N,N-di(C₁-C₄ allyl)-amino (preferably N,N-dimethylamino or N,N-diethylamino), sulfonyl, C₁-C₄ alkylsulfonyl (preferably methylsulfonyl or ethylsulfonyl), sulfinyl and C₁-C₄ alkylsulfinyl (preferably methylsulfinyl); heteroaryl; heterocyclyl; C₁-C₇ alkoxy; C₁-C₇ alkylthio; C₁-C₇ alkanoyl; aryloyl; oxo-C₁-C₇ alkyl; C₁-C₇ alkanoyloxy; carboxy; C₁-C₇ alkyloxycarbonyl which is unsubstituted or is substituted by 1 to 3 substituents selected from the group consisting of halogen (preferably fluorine or chlorine), hydroxy, C₁-C₄ alkoxy (preferably methoxy or ethoxy), C₁-C₄ alkoxycarbonyl (preferably methoxycarbonyl or ethoxycarbonyl), thiol, C₁-C₄ alkylthio (preferably methylthio or ethylthio), amino, N—(C₁-C₄) alkylamino (preferably N-methylamino or N ethylamino), N,N-di(C₁-C₄ alkyl)-amino (preferably dimethylamino or diethylamino), sulfonyl, C₁-C₄ alkylsulfonyl (preferably methylsulfonyl or ethylsulfonyl), sulfinyl and C₁-C₄ alkylsulfinyl (preferably methylsulfinyl); or C₇-C₁₀ aryloxycarbonyl which is unsubstituted or is substituted by 1 to 3 substituents selected from the group consisting of halogen (preferably fluorine or chlorine), hydroxy, C₁-C₄ alkoxy (preferably methoxy or ethoxy), C₁-C₄ alkoxycarbonyl (preferably methoxycarbonyl or ethoxycarbonyl), thiol, C₁-C₄ alkylthio (preferably methylthio or ethylthio), amino, N—(C₁-C₄) alkylamino (preferably N-methylamino or N ethylamino), N,N-di(C₁-C₄ alkyl)-amino (preferably dimethylamino or diethylamino), sulfonyl, C₁-C₄ alkylsulfonyl (preferably methylsulfonyl or ethylsulfonyl), sulfinyl and C₁-C₄ alkylsulfinyl (preferably methylsulfinyl); carbamoyl; N—(C₁-C₇ alkyl)carbamoyl; N,N-di(C₁-C₇-alkyl)carbamoyl; cyano-C₁-C₇ alkyl; C₁-C₇ alkylsulfonyl; C₁-C₇ alkylsulfinyl;
   n and p are each independently integers from 0 to 5 with proviso that, when n has the meaning of zero, Q1 and Q2 cannot simultaneously be oxygen, sulfur or

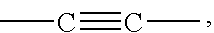

and when p has the meaning of zero, Q2 cannot be oxygen, sulfur or

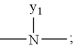

R² is halogen (preferably chlorine or fluorine);

R³, R⁴ and R⁵ are independently selected from hydrogen, halo, C₁-C₇ alkyl which is unsubstituted or is substituted by 1 to 3 substituents selected from the group consisting of halogen (preferably fluorine or chlorine), hydroxy, C₁-C₄ alkoxy (preferably methoxy or ethoxy), C₁-C₄ alkoxycarbonyl (preferably methoxycarbonyl or ethoxycarbonyl), thiol, C₁-C₄ alkylthio (preferably methylthio or ethylthio), amino, N—(C₁-C₄) alkylamino (preferably N-methylamino or N ethylamino), N,N-di(C₁-C₄ alkyl)-amino (preferably dimethylamino or diethylamino), sulfonyl, C₁-C₄ alkylsulfonyl (preferably methylsulfonyl or ethylsulfonyl), sulfinyl and C₁-C₄ alkylsulfinyl (preferably methylsulfinyl), hydroxy, C₁-C₇ alkoxy, thiol, C₁-C₇ alkylthio, amino, N—(C₁-C₇ alkyl)amino, N,N-di(C₁-C₇ alkyl)amino, C₁-C₇ alkanoyloxy, carboxy, C₁-C₇ alkyloxycarbonyl which is unsubstituted or is substituted by 1 to 3 substituents selected from the group consisting of halogen (preferably fluorine or chlorine), hydroxy, C₁-C₄ alkoxy (preferably methoxy or ethoxy), C₁-C₄ alkoxycarbonyl (preferably methoxycarbonyl or ethoxycarbonyl), thiol, C₁-C₄ alkylthio (preferably methylthio or ethylthio), amino, N—(C₁-C₄) alkylamino (preferably N-methylamino or N ethylamino), N,N-di(C₁-C₄ alkyl)-amino (preferably dimethylamino or diethylamino), sulfonyl, C₁-C₄ alkylsulfonyl (preferably methylsulfonyl or ethylsulfonyl), sulfinyl and C₁-C₄ alkylsulfinyl (preferably methylsulfinyl), C₇-C₁₀ aryloxycarbonyl which is unsubstituted or is substituted by 1 to 3 substituents selected from the group consisting of halogen (preferably fluorine or chlorine), hydroxy, C₁-C₄ alkoxy (preferably methoxy or ethoxy), thiol, C₁-C₄ alkoxycarbonyl (preferably methoxycarbonyl or ethoxycarbonyl), C₁-C₄ alkylthio (preferably methylthio or ethylthio), amino, N—(C₁-C₄) alkylamino (preferably N-methylamino or N ethylamino), N,N-di(C₁-C₄ alkyl)-amino (preferably dimethylamino or diethylamino), sulfonyl, C₁-C₄ alkylsulfonyl (preferably methylsulfonyl or ethylsulfonyl), sulfinyl and C₁-C₄ alkylsulfinyl (preferably methylsulfinyl), carbamoyl, N—(C₁-C₇-alkyl)carbamoyl, N,N-di(C₁-C₇-alkyl)carbamoyl, cyano, cyano-C₁-C₇ alkyl, sulfonyl, C₁-C₇ alkylsulfonyl, sulfinyl, C₁-C₇ alkylsulfinyl or nitro group; and to pharmacologically acceptable derivatives, e.g., salts or solvates, thereof; and wherein all preferences are satisfied simultaneously or serially, preferably simultaneously.

The present invention also relates to pharmaceutical compositions comprising the compounds of Formula I and a pharmaceutically acceptable carrier.

The present invention also relates to compositions containing one or more of the compounds Formula I in an amount effective to treat central nervous system (CNS) and other disorders that are related to imbalance of monoamine neurotransmitters in mammals, including humans.

The present invention further relates to methods for using the compounds of Formula I to treat central nervous system (CNS) and other disorders that are related to imbalance of monoamine neurotransmitters in mammals, including humans.

The present invention additionally relates to a method of treating a central nervous system (CNS) disorder and/or a disorder that is related to imbalance of monoamine neurotransmitters, comprising administration of a compound of Formula I to a patient in need thereof.

### DETAILED DESCRIPTION OF THE INVENTION

No compound representing the subject of the present invention has been described as a reuptake inhibitor of a monoamine-derived neurotransmitter, i.e. dopamine, serotonin and norepinephrine. Consequently, the use of such compounds and pharmaceutical compositions containing them to treat the disorders that are related to imbalance of monoamine neurotransmitters in CNS as well as to treat conditions and states that involve different types of pain has not been described or suggested. Moreover, there has not been a description or suggestion of pharmaceutical dosage forms containing effective amounts of any tetracyclic dibenzo(e,h)azulene compounds to treat monoamine neurotransmitter-based central nervous system (CNS) disorders in a mammalian subject, including the human.

In one embodiment of the present invention are compounds of Formula I those wherein X represents O or S.

In another embodiment of the invention are the compounds of Formula I and to pharmaceutically acceptable derivatives thereof wherein R¹ is represented by Formula II:

Q₁-(CH₂)n-Q₂-(CH₂)p-A II
wherein Q₁ and Q₂ are independently selected from oxygen, sulfur or group selected from:

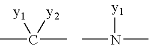

wherein Y₁ and Y₂ are independently selected from hydrogen; C₁-C₄ alkyl
A is selected from amino, N—(C₁-C₇ alkyl)amino, N,N-di(C₁-C₇ alkyl)amino, heterocyclic or heteroaryl group selected from the group consisting of morpholine-4-yl, piperidine-1-yl, pyrrolidine-1-yl, imidazole-1-yl and piperazine-1-yl, or represented by the structure of Formula III:

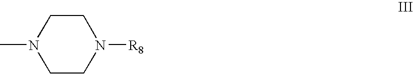

wherein R⁸ is selected from hydrogen; C₁-C₇ alkyl which is unsubstituted or is substituted as defined above; C₁-C₇ alkoxy; C₁-C₇ alkylthio; C₁-C₇ alkanoyl; aryloyl; oxo-C₁-C₇ alkyl; C₁-C₇ alkanoyloxy; carboxy; C₁-C₇ alkyloxycarbonyl which is unsubstituted or is substituted by; or C₇-C₁₀ aryloxycarbonyl which is unsubstituted or is substituted by; carbamoyl; N—(C₁-C₇ alkyl)carbamoyl; N,N-di(C₁-C₇-allyl)carbamoyl; cyano-C₁-C₇ alkyl; C₁-C₇ alkylsulfonyl; C₁-C₇ alkylsulfinyl;
   n and p are each independently integers from 0 to 5 with proviso that when n has the meaning of zero, Q1 and Q2 cannot simultaneously be oxygen, sulfur or

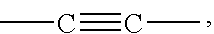

and when p has the meaning of zero, Q2 cannot be oxygen, sulfur or

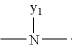

In yet another embodiment of the invention are the compounds of Formula I,

wherein R⁴ and R⁵ are independently selected from hydrogen, halo, C₁-C₇ alkyl which is unsubstituted or is substituted as specified above, hydroxy, C₁-C₇ alkoxy, thiol, C₁-C₇ alkylthio and R3 represents C₁-C₇ alkanoyloxy, carboxy, C₁-C₇ alkyloxycarbonyl which is unsubstituted or is substituted by, C₇-C₁₀ aryloxycarbonyl which is unsubstituted or is substituted by, carbamoyl, N—(C₁-C₇-alkyl)carbamoyl, N,N-di(C₁-C₇-alkyl)carbamoyl, cyano group; and pharmacologically acceptable salts and solvates thereof.

In one particular embodiment are the compounds of Formula I and to pharmaceutically acceptable derivatives thereof wherein

### X is —O—

W is an aromatic —CH—

### Z is —S—

R¹ is represented by Formula II:

Q₁-(CH₂)n-Q₂-(CH₂)p-A; wherein

Q₁ is oxygen;

### Q₂ is

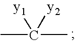

wherein
Y₁ and are hydrogen, and
   A is N,N-di(C₁-C₂ alkyl)amino;
   n and p are 1
   R² is chlorine;
   R³ is hydroxymethyl, acetylmethyl, aminocarbonyl, dimethylaminocarbonyl, diethylaminocarbonyl, or ethoxycarbonyl; and
   R⁴ and R⁵ are hydrogen.

In another particular embodiment are compounds of Formula I, wherein the compounds are 2-Substituted-1-chloro-9-(3-dimethylaminopropoxy)-8-oxa-1-thia-dibenzo[e,h]azulenes, wherein R3 represents C₁-C₇ alkyloxycarbonyl, carbamoyl, N—(C₁-C₇-alkyl)carbamoyl, N,N-di(C₁-C₇-alkyl)carbamoyl, cyano group; and pharmacologically acceptable salts and solvates thereof.

Particularly preferred compounds of the invention are:
11-Chloro-9-(3-dimethylaminopropoxy)-8-oxa-1-thia-dibenzo[e,h]azulene-2-carboxylic acid ethyl ester;
2-Hydroxymethyl-11-Chloro-9-(3-diethylaminopropoxy)-8-oxa-1-thia-dibenzo[e,h]azulene;
11-Chloro-9-(3-dimethylaminopropoxy)-8-oxa-1-thia-dibenzo[e,h]azulene-2-ylmethyl acetate;
11-Chloro-9-(3-dimethylaminopropoxy)-8-oxa-1-thia-dibenzo[e,h]azulene-2-carboxylic acid amide;
N,N-Dimethyl-11-chloro-9-(3-dimethylaminopropoxy)-8-oxa-1-thia-dibenzo[e,h]azulene-2-carboxamide;
N,N-Diethyl-11-chloro-9-(3-dimethylaminopropoxy)-8-oxa-1-thia-dibenzo[e,h]azulene-2-carboxamide; and
   pharmaceutically acceptable derivatives thereof.

### Salts, Solvates, Prodrugs, and Stereoisomers

Typically, a pharmaceutically acceptable salt of a compound of Formula I may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of Formula I and the resulting mixture evaporated to dryness (lyophilized) to obtain the acid addition salt as a solid. Alternatively, a compound of Formula I may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the compounds of Formula I may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

Compounds of the invention may have both a basic and an acidic center may and therefore be in the form of zwitterions.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention. The salts of the compound of Formula I may form solvates (e.g. hydrates) and the invention also includes all such solvates.

The present invention also encompasses prodrugs of Formula I, i.e., compounds which release an active parent drug according to Formula I in vivo when administered to a mammalian subject. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of Formula I. Further, in the case of a carboxylic acid (—COOH), esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

The compounds of Formula I may exist in numerous forms of structural isomers that may be formed as a result of tautomerism, and may exist in different ratios at equilibrium. Due to dynamic equilibrium such isomers (tautomers) are rapidly interconvertible from one isomeric form to another. The most common isomerism is keto-enol tautomerism, but equilibrium between open chain and cyclic forms are also known. It is to be understood that whenever in the present invention we refer to the compounds of Formula I we mean to include tautomeric forms thereof, keto-enol tautomeric, open chain-cyclic, isolated as separate isomers or existing in any other mixture of different ratios at equilibrium. The isomeric forms predominant for a particular compound of Formula I are dependent on the nature of the substituent, whether the compound exists in the free form or in the form of any of its salts, type of the salt, solvent in which the compound is dissolved, as well as pH value of the solution.

Compounds of the present invention may further exist as different geometric isomers or different stereoisomers. Isomers that differ only with regard to the arrangement of the atoms in the space around the asymmetric (stereogenic, chiral) center are called “stereoisomers”. Stereoisomers that are not mirror images of each other are called diastereomers, while stereoisomers that have a mirror-image relationship, i.e. that are mirror images of each other, are called enantiomers. Each stereoisomer may be characterized by determining the absolute configuration of the stereogenic center by the use of Cahn-Ingold-Prelog priority rules and hence characterized as the R- or S-isomer. Another way of identification of stereoisomers is the measurement of the rotation of the plane of polarized light that passes through the molecule, and designating chiral molecules to be right-rotating (+) or left-rotating (−) isomers. Chiral molecules may exist in a form of single enantiomer or in a mixture of enantiomers. A mixture consisting of equal parts (+) and (−) enantiomers of a chiral substance is called racemic mixture. The present invention relates to each stereoisomer that may be shown by Formula I either isolated as separate enantiomers, diastereomers or existing in racemic or any other mixture thereof.

Methods for determination of stereochemical configuration, resolution and separation of stereoisomers are well known from the literature. The enantiomers may be resolved by methods known to those skilled in the art, for example by formation of diastereomeric salts which may be separated, for example, by crystallization; formation of diastereomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. The diastereomeric pairs may be separated by methods known to those skilled in the art, for example chromatography or crystallization and the individual enantiomers within each pair may be separated as described above.

The present invention also encompasses stereoisomers of the syn-anti type, and mixtures thereof encountered when an oxime or similar group is present. The group of highest Cahn-Ingold-Prelog priority attached to one of the terminal doubly bonded atoms of the oxime, is compared with hydroxy group of the oxime. The stereoisomer is designated as Z (zusammen=together) or Syn if the oxime hydroxyl lies on the same side of a reference plane passing through the C═N double bond as the group of highest priority; the other stereoisomer is designated as E (entgegen=opposite) or Anti.

The compounds of Formula I may be in crystalline or amorphous form. Furthermore, some of the crystalline forms of the compounds of Formula I may exist as polymorphs, which are included in the present invention.

The present invention also relates to all radiolabelled forms of the compounds of Formula I. Preferred radiolabelled compounds of Formula I are those wherein the radiolabels are selected from as ³H, ¹¹C, ¹⁴C, and ¹⁸F. Such radiolabelled compounds are useful as research and diagnostic tools in metabolism pharmacokinetic studies and in binding assays in both animals and man.

### DEFINITIONS

The term “halo”, “hal” or “halogen” relates to a fluorine, chlorine, bromine or iodine atom (preferably chlorine or bromine).

The term “alkyl” as used herein as a group or a part of a group refers to a straight or branched hydrocarbon chain containing the specified number of carbon atoms. Thus the term “alkyl” relates to alkyl groups with the meaning of alkanes wherefrom radicals are derived, which radicals may be straight, branched or cyclic or a combination of straight and cyclic ones and branched and cyclic ones. For example, C₁-C₇ alkyl means a straight or branched alkyl chain containing from 1 to 7 carbon atoms; examples of such group include methyl, ethyl, propyl, isopropyl, butyl, sec-butyl and tert-butyl. The preferred cyclic alkyls are e.g. cyclopentyl or cyclohexyl.

The term “haloalkyl” relates to alkyl groups which must be substituted with at least one halogen atom. The most frequent haloalkyls are e.g. chloromethyl, dichloromethyl, trifluoromethyl or 1,2-dichloropropyl.

The term “alkenyl” as used herein as a group or a part of a group refers to a alkenyl groups having the meaning of hydrocarbon radicals containing the specified number of carbon atoms, which may be straight, branched or cyclic or are a combination of straight and cyclic ones or branched and cyclic ones, but having at least one carbon-carbon double bond. For example, the term “C₂-C₇ alkenyl” means a straight, branched or cyclic alkenyl containing at least 2, and at most 7, carbon atoms and containing at least one double bond. Examples of “alkenyl” as used herein include, but are not limited to, ethenyl, 2-propenyl, 3-butenyl, 2-butenyl, 2-pentenyl, 3-pentenyl, 3-methyl-2-butenyl, 3-methylbut-2-enyl, 3-hexenyl and 1,1-dimethylbut-2-enyl or cyclohexenyl. The most frequent alkenyls are ethenyl, propenyl, butenyl or cyclohexenyl. It will be appreciated that in groups of the form —O—C₂-C₇ alkenyl, the double bond is preferably not adjacent to the oxygen.

The term “alkynyl” as used herein as a group or a part of a group refers to a straight or branched hydrocarbon chain containing the specified number of carbon atoms and containing at least one triple bond. For example, the term “C₃-C₇ alkenyl” means a straight or branched alkynyl containing at least 3, and at most 7, carbon atoms containing at least one triple bond. Examples of “alkynyl” as used herein include, but are not limited to, ethynyl, propynyl, 1-butynyl, 2-butynyl, 1-pentynyl and 3-methyl-1-butynyl. The most frequent alkynyls are e.g. ethynyl, propynyl or butynyl.

The term “alkoxy” as used herein refers to a straight or branched chain alkoxy group containing the specified number of carbon atoms. For example, C₁-C₇ alkoxy means a straight or branched alkoxy containing at least 1, and at most 7, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy.

The term “aryl” as used herein as a group or a part of a group refers to an aromatic carbocyclic moiety containing one ring with at least 6 carbon atoms or two rings with totally 10 carbon atoms and with alternating double (resonant) bonds between carbon atoms. The most frequently used aryls are e.g. phenyl or naphthyl. In general, aryl groups may be linked to the rest of the molecule by any available carbon atom via a direct bond or via a C₁-C₄ alkylene group such as methylene or ethylene.

The term “heteroaryl” as used herein, unless otherwise defined, relates to a monocyclic or bicyclic ring with 4 to 12 carbon atoms which is aromatic or partially aromatic, having at least one hetero atom selected from nitrogen, oxygen and sulfur, wherein the “heteroaryl” group may form a linkage to the rest of the molecule through the available nitrogen or carbon atom either via a direct bond or via a C₁-C₄ alkylene group defined earlier. Examples of heteroaryl group include, but are not limited to thiophenyl, pyrrolyl, imidazolyl, pyridinyl, oxazolyl, thiazolyl, pyrazolyl, tetrazolyl, pyrimidinyl, pyrazinyl, quinolinyl or triazinyl.

The term “heterocyclyl” as used herein, unless otherwise defined, relates to five-member or six-member, fully saturated or partly unsaturated heterocyclic group containing at least one hetero atom selected from oxygen, nitrogen and sulfur, wherein the “heterocyclyl” group may form a linkage to the rest of the molecule through the available nitrogen or carbon atom either via a direct bond or via a C₁-C₄ alkylene group defined earlier. Examples of heterocyclyl group include, but are not limited to morpholinyl, piperidinyl, piperazinyl, pyrrolidinyl, pyrazinyl or imidazolyl.

The term “C₁-C₇ alkanoyl” refers to acyl group such as formyl, acetyl, propanoyl or butanoyl.

The term “aryloyl” as used herein, unless otherwise defined, relates to aromatic acyl groups such as benzoyl.

The term “optionally substituted alkyl” or “substituted alkyl” as used herein relates to an alkyl group which is substituted by one, two, three or more substituents. selected from the group consisting of halogen atom (preferably fluorine or chlorine), hydroxy, C₁-C₄ alkoxy (preferably methoxy or ethoxy), thiol, C₁-C₄ alkylthio (preferably methylthio or ethylthio), amino, N—(C₁-C₄)alkylamino (preferably N-methylamino or N ethylamino), N,N-di(C₁-C₄-alkyl)-amino (preferably dimethylamino or diethylamino), sulfonyl, C₁-C₄ alkylsulfonyl (preferably methylsulfonyl or ethylsulfonyl), sulfinyl, C₁-C₄ alkylsulfinyl (preferably methylsulfinyl).

The term “optionally substituted alkenyl” or “substituted alkenyl” as used herein relates to an alkenyl group substituted by one, two or three halogen atoms. Such substituents may be e.g. 2-chloroethenyl, 1,2-dichloroethenyl or 2-bromo-propene-1-yl.

The term “optionally substituted aryl, heteroaryl or heterocycle” or “substituted aryl, heteroaryl or heterocycle” as used herein relates to an aryl, heteroaryl or heterocyclic group which is substituted by one or two substituents selected from the group selected from halogen (preferably chlorine or fluorine), C₁-C₄ alkyl (preferably methyl, ethyl or isopropyl), cyano, nitro, hydroxy, C₁-C₄ alkoxy (preferably methoxy or ethoxy), thiol, C₁-C₄ alkylthio (preferably methylthio or ethylthio), amino, N—(C₁-C₄) alkylamino (preferably N-methylamino or N-ethylamino), N,N-di(C₁-C₄-alkyl)-amino (preferably N,N-dimethylamino or N,N-diethylamino), sulfonyl, C₁-C₄ alkylsulfonyl (preferably methylsulfonyl or ethylsulfonyl), sulfinyl, C₁-C₄ alkylsulfinyl (preferably methylsulfinyl).

As used herein, the term “salts” can include acid addition salts or addition salts of free bases. Examples of acids which may be employed to form pharmaceutically acceptable acid addition salts include but are not limited to salts derived from nontoxic inorganic acids such as nitric, phosphoric, sulfuric, or hydrobromic, hydroiodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al. “Pharmaceutical Salts,” J. of Pharma. Sci., 1977; 66:1).

The term “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “carrier” applied to pharmaceutical compositions of the invention refers to a diluent, excipient, or vehicle with which an active compound is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. However, since benzodiazulene salt are highly soluble, aqueous solutions are preferred. Suitable pharmaceutical carriers are described in “Remington's Pharmaceutical Sciences” by E. W. Martin, 18th Edition. Particularly preferred for the present invention are carriers suitable for immediate-release, i.e., release of most or all of the active ingredient over a short period of time, such as 60 minutes or less, and make rapid absorption of the drug possible.

The term “pharmaceutically acceptable derivative” as used herein means any pharmaceutically acceptable salt, solvate or prodrug, e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol 1: Principles and Practice, which is incorporated herein by reference to the extent of teaching such derivatives. Preferred pharmaceutically acceptable derivatives are salts, solvates, esters, carbamates and phosphate esters. Particularly preferred pharmaceutically acceptable derivatives are salts, solvates and esters. Most preferred pharmaceutically acceptable derivatives are salts and esters.

The term “prodrug” as used herein means a compound which is converted within the body, e.g. by hydrolysis in the blood, into its active form that has medical effects. Pharmaceutically acceptable prodrugs are described in T. Higuchi and V. Stella, “Prodrugs as Novel Delivery Systems”, Vol. 14 of the A.C.S. Symposium Series, Edward B. Roche, ed., “Bioreversible Carriers in Drug Design”, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

As used herein, “treating” or “treatment” of a state, disorder or condition includes:

(1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition,
(2) inhibiting the state, disorder or condition, i.e., arresting, reducing or delaying the development of the disease or a relapse thereof (in case of maintenance treatment) or at least one clinical or subclinical symptom thereof, or
(3) relieving or attenuating the disease, i.e., causing regression of the state, disorder or condition or at least one of its clinical or subclinical symptoms.

The benefit to a subject to be treated is either statistically significant or at least perceptible to the patient or to the physician.

“Patient” refers to mammals, preferably humans or domestic animals, more preferably humans.

As used herein, a “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

As used herein, “delivering” a therapeutically effective amount of an active ingredient to a particular location within a host means causing a therapeutically effective blood concentration of the active ingredient at the particular location. This can be accomplished, e.g., by local or by systemic administration of the active ingredient to the host.

The term “host” or “subject in need thereof” as used herein refers to a mammal preferably a human.

“Chemical dependency.” as used herein, means an abnormal craving or desire for or an addiction to a drug. Such drugs are generally administered to the affected individual by any of a variety of means of administration, including oral, parenteral, nasal or by inhalation. Examples of chemical dependencies treatable by the methods of the present invention are dependencies on alcohol, nicotine, cocaine, heroin, phenolbarbitol, and benzodiazepines (e.g., Valium®). “Treating a chemical dependency,” as used herein, means reducing or alleviating such dependency.

References hereinafter to a compound according to the invention include both compounds of Formula I and their pharmaceutically acceptable derivatives.

### Pharmaceutical Compositions

While it is possible that, for use in therapy, a compound of the invention may be administered as the raw chemical, it is preferable to present the active ingredient in a pharmaceutical formulation, e.g., when the agent is in admixture with a suitable pharmaceutical excipient, diluent or carrier selected with regard to the intended route of administration and standard pharmaceutical practice.

Accordingly, in one aspect, the present invention provides a pharmaceutical composition or formulation comprising at least one compound of the invention or a pharmaceutically acceptable derivative thereof in association with a pharmaceutically acceptable excipient, diluent and/or carrier. The excipient, diluent and/or carrier must be “acceptable” in the sense of being compatible with the other ingredients of Formulation and not deleterious to the recipient thereof.

In another aspect, the invention provides a pharmaceutical composition comprising, as active ingredient, at least one compound of the invention or a pharmaceutically acceptable derivative thereof in association with a pharmaceutically acceptable excipient, diluent and/or carrier for use in therapy, and in particular, in the treatment of human or animal subjects suffering from a condition susceptible to amelioration by an antimicrobial compound.

In another aspect, the invention provides a pharmaceutical composition comprising a therapeutically effective amount of the compounds of the present invention and a pharmaceutically acceptable excipient, diluent and/or carrier (including combinations thereof).

The compounds of the invention may be Formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable excipients, diluents and/or carriers. Acceptable excipients, diluents and carriers for therapeutic use are well known in the pharmaceutical art, and are described, for example, in Remington's Pharmaceutical Sciences, Mack Publishing Co. (A. R. Gennaro edit. 1985). The choice of pharmaceutical excipient, diluent and/or carrier can be selected with regard to the intended route of administration and standard pharmaceutical practice. The pharmaceutical compositions may comprise as—or in addition to—the excipient, diluent and/or carrier any suitable binder(s), lubricant(s), suspending agent(s), coating agent(s), solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubilizer. Alpha-, beta- and gamma-cyclodextrins are most commonly used and suitable examples are described in WO 91/11172, WO 94/02518 and WO 98/55148.

The compounds of the invention may be milled using known milling procedures such as wet milling to obtain a particle size appropriate for tablet formation and for other Formulation types. Finely divided (nanoparticulate) preparations of the compounds of the invention may be prepared by processes known in the art, for example see International Patent Application No. WO 02/00196 (SmithKline Beecham).

The routes for administration (delivery) include, but are not limited to, one or more of: oral (e.g. as a tablet, capsule, or as an ingestible solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

There may be different composition/formulation requirements depending on the different delivery systems. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intravenously, intramuscularly or subcutaneously. For parenteral administration, the compositions may be best used in the form of a sterile aqueous solution, which may contain other substances, for example enough salts or monosaccharides to make the solution isotonic with blood. For buccal or sublingual administration the compositions may be administered in the form of tablets or lozenges, which can be Formulated in a conventional manner.

It is to be understood that not all of the compounds need be administered by the same route. Likewise, if the composition comprises more than one active component, then those components may be administered by different routes.

The compositions of the invention include those in a form especially formulated for parenteral, oral, buccal, rectal, topical, implant, ophthalmic, nasal or genito-urinary use. For some applications, the agents of the present invention are delivered systemically (such as orally, buccally, sublingually), more preferably orally. Hence, preferably the agent is in a form that is suitable for oral delivery.

If the compound of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the agent; and/or by using infusion techniques.

For parenteral administration, the compound is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral Formulations under sterile conditions is readily accomplished by standard pharmaceutical techniques well-known to those skilled in the art.

The compounds according to the invention may be formulated for use in human or veterinary medicine by injection (e.g. by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain Formulatory agents such as suspending, stabilizing, solubilizing and/or dispersing agents. Alternatively the active ingredient may be in sterile powder form for reconstitution with a suitable vehicle, e.g. sterile, pyrogen-free water, before use.

The compounds of the invention can be administered (e.g. orally or topically) in the form of tablets, capsules, ovules, elixirs, solutions or suspensions, which may contain flavoring or coloring agents, for immediate-, delayed-, modified-, sustained-, pulsed- or controlled-release applications.

The compounds of the invention may also be presented for human or veterinary use in a form suitable for oral or buccal administration, for example in the form of solutions, gels, syrups, mouth washes or suspensions, or a dry powder for constitution with water or other suitable vehicle before use, optionally with flavoring and coloring agents. Solid compositions such as tablets, capsules, lozenges, pastilles, pills, boluses, powder, pastes, granules, bullets or premix preparations may also be used. Solid and liquid compositions for oral use may be prepared according to methods well known in the art. Such compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

The compounds of the invention may also be administered orally in veterinary medicine in the form of a liquid drench such as a solution, suspension or dispersion of the active ingredient together with a pharmaceutically acceptable carrier or excipient.

The compounds of the invention may also, for example, be formulated as suppositories e.g. containing conventional suppository bases for use in human or veterinary medicine or as pessaries e.g. containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g. eye ear or nose drops) or pour-ons.

For application topically to the skin, the agent of the present invention can be Formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water.

Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds may also be dermally or transdermally administered, for example, by use of a skin patch.

For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134AT″″) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g. using a mixture of ethanol and the propellant as the solvent, which may additionally contain a lubricant, e.g. sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebulizer.

The compounds of the invention may also be used in combination with other therapeutic agents. The invention thus provides, in a further aspect, a combination comprising a compound of the invention or a pharmaceutically acceptable derivative thereof together with a further therapeutic agent.

When a compound of the invention or a pharmaceutically acceptable derivative thereof is used in combination with a second therapeutic agent active against the same disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may for example be used for topical administration with other active ingredients such as corticosteroids or antifungals as appropriate.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical Formulations by any convenient route.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of formulation. When formulated separately they may be provided in any convenient formulation, conveniently in such manner as are known for such compounds in the art.

The compositions may contain from 0.01-99% of the active material. For topical administration, for example, the composition will generally contain from 0.01-10%, more preferably 0.01-1% of the active material.

The preparation of pharmaceutical formulations may include blending, granulating, tabletting and dissolving the ingredients. Pharmaceutically acceptable carriers (binders and fillers) may be solid or liquid. Solid carriers may be lactose, sucrose, talcum, gelatine, agar, pectin, magnesium stearate, fatty acids etc. Liquid carriers may be syrups, oils such as olive oil, sunflower oil or soy bean oil, water etc. Similarly, the pharmaceutically acceptable Formulations may also contain a component for a sustained release of the active component such as e.g. glyceryl monostearate or glyceryl distearate. Various forms of pharmaceutical Formulations may be used. Thus, if a solid carrier is used, these forms may be tablets, hard gelatine capsules, powder or granules, which may be administered in capsules per os. The amount of the solid carrier may vary, but it is mainly from 25 mg to 1 g. If a liquid carrier is used, Formulation would be in the form of a syrup, emulsion, soft gelatine capsules, sterile injectable liquids such as ampoules or non-aqueous liquid suspensions.

The present invention also relates to a pharmaceutical composition for treating a disorder or condition selected from depression (e.g., depression in cancer patients, depression in Parkinson's patients. postmyocardial infarction depression, subsyndromal symptomatic depression, depression in infertile women. pediatric depression, major depression, single episode depression, recurrent depression, child abuse induced depression, and post partum depression), generalized anxiety disorder, phobias (e.g., agoraphobia, social phobia and simple phobias), post-traumatic stress syndrome, avoidant personality disorder, premature ejaculation, eating disorders (e.g., anorexia nervosa and bulimia nervosa), obesity, chemical dependencies (e.g., addictions to alcohol, cocaine, heroin, phenobarbital, nicotine and benzodiazepines), cluster headache, migraine, pain, Alzheimer's disease, obsessive-compulsive disorder, panic disorder, memory disorders (e.g. dementia. amnesic disorders, and age-related cognitive decline (ARCD)), Parkinson's diseases (e.g. dementia in Parkinson's disease, neuroleptics-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, attention deficit hyperactivity disorder (ADHD), chronic paroxysmal hemicrania and headache (associated with vascular disorders) in a mammal, preferably a human, comprising an amount of a compound of Formula I or a pharmaceutically acceptable salt thereof effective in treating such disorder or condition and a pharmaceutically acceptable carrier.

The present invention also relates to a pharmaceutical composition for treating a disorder or condition that can be treated by inhibiting the reuptake of serotonin, dopamine or norepinephrine in a mammal, preferably a human, comprising an amount of a compound of Formula I, or a pharmaceutically acceptable salt thereof, that is effective in treating such disorder or condition and a pharmaceutically acceptable carrier. Examples of such disorders and conditions are those enumerated in the preceding paragraph.

The present invention also relates to a method for treating a disorder or condition selected from depression (e.g., depression in cancer patients, depression in Parkinson's patients, postmyocardial infarction depression, subsyndromal symptomatic depression, depression in infertile women, pediatric depression, major depression, single episode depression, recurrent depression, child abuse induced depression, and post partum depression), generalized anxiety disorder, phobias (e.g., agoraphobia, social phobia and simple phobias), post-traumatic stress syndrome, avoidant personality disorder, premature ejaculation, eating disorders (e.g., anorexia nervosa and bulimia nervosa), obesity, chemical dependencies (e.g., addictions to alcohol, cocaine, heroin, phenobarbital, nicotine and benzodiazepines), cluster headache, migraine, pain, Alzheimer's disease, obsessive-compulsive disorder, panic disorder, memory disorders (e.g., dementia, amnesic disorders, and age-related cognitive decline (ARCD), Parkinson's diseases (e.g., dementia in Parkinson's disease, neuroleptics-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, attention deficit hyperactivity disorder (ADHD), chronic paroxysmal hemicrania and headache (associated with vascular disorders) in a mammal, preferably a human, comprising administering to a mammal in need of such treatment an amount of a compound of Formula I, or a pharmaceutically acceptable salt thereof, that is effective in treating such disorder or condition.

The present invention also relates to a method for treating a disorder or condition that can be treated by inhibiting the reuptake of serotonin, dopamine or norepinephrine in a mammal, preferably a human, comprising administering to a mammal in need of such treatment an amount of a compound of Formula I, or a pharmaceutically acceptable salt thereof, that is effective in treating such disorder or condition.

The present invention relates to a pharmaceutical composition for treating a condition or disorder that can be treated by inhibiting the reuptake of serotonin, dopamine or norepinephrine in a mammal, preferably a human, comprising:

a) a pharmaceutically acceptable carrier; and
b) a compound of Formula I or a pharmaceutically acceptable salt or solvate thereof,
wherein the amount of the active compound of Formula I is effective in treating such disorder or condition.

The present invention also relates to a method for treating a disorder or condition that can be treated by inhibiting the reuptake of serotonin, dopamine or norepinephrine in a mammal, preferably a human, comprising administering to a mammal requiring such treatment, a compound of Formula I or a pharmaceutically acceptable derivative thereof, wherein the amounts of the active compounds (e.g., the compound of Formula I is such that the amount is effective in treating such disorder or condition.

### Dosages

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

For oral and parenteral administration to humans, the daily dosage level of the agent may be in single or divided doses.

For systemic administration the daily dose as employed for adult human treatment it will range from 1-5000 μg/kg body weight, preferably 10-100 μg/kg body weight, which may be administered in 1 to 4 daily doses, for example, depending on the route of administration and the condition of the patient. When the composition comprises dosage units, each unit will preferably contain 1 mg to 100 mg of active ingredient. The duration of treatment will be dictated by the rate of response rather than by arbitrary numbers of days.

Further, the present invention relates to a pharmaceutical formulation containing an effective non-toxic dose of the compound of Formula I as well as pharmaceutically acceptable carriers or solvents.

In another embodiment of the present invention the compound of Formula I, or pharmaceutically acceptable derivative thereof, show binding affinity to serotonin, dopamine and norepinephrine transporters in the concentration expressed as an IC₅₀ value less than 1 μM and having Kᵢ value less than 1 μM.

### Methods of Preparation of the Active Compounds:

Compounds of Formula (I) and pharmaceutically acceptable derivatives thereof may be prepared by the general methods outlined hereinafter, said methods constituting a further aspect of the invention. In the following description, the groups R¹ to R⁸, A, Q₁, Q₂, X, W, Z, y₁, y₂, n and p have the meaning defined for the compounds of Formula I unless otherwise stated.

It will be appreciated by those skilled in the art that it may be desirable to use protected derivatives of intermediates used in the preparation of the compounds of Formula I. Protection and deprotection of functional groups may be performed by methods known in the art. Hydroxyl or amino groups may be protected with any hydroxyl or amino protecting group, for example, as described in Green T. W.; Wuts P. G. M. Protective Groups in Organic Synthesis: John Wiley and Sons, New York, 1999. The amino protecting groups may be removed by conventional techniques. For example, acyl groups, such as alkanoyl, alkoxycarbonyl and aryloyl groups, may be removed by solvolysis, e.g., by hydrolysis under acidic or basic conditions. Arylmethoxycarbonyl groups (e.g., benzyloxycarbonyl) may be cleaved by hydrogenolysis in the presence of a catalyst such as palladium-on-charcoal.

The synthesis of the target compound is completed by removing any protecting groups, which are present in the penultimate intermediate using standard techniques, which are well known to those skilled in the art. The deprotected final product is then purified, as necessary, using standard techniques such as silica gel chromatography, HPLC on silica gel, and the like or by recrystallization.

A further object of the present invention relates to the preparation of compounds of Formula I according to processes comprising:

(a) For compounds of Formula I, wherein Q₁ has the meaning of —O—, —S—,

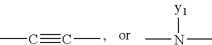

a reaction of alcohols of Formula IVa:

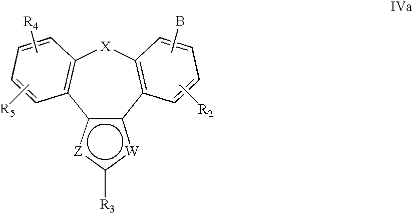

wherein B represents —OH, Ny₁H, SH, or

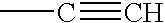

with compounds of Formula Va,

L-(CH₂)n-Q₂-(CH₂)p-A Va

wherein L is a leaving group which may be a halogen atom (most frequently bromine, iodine or chlorine) or sulfonyloxy group (most frequently trifluoromethylsulfonyloxy (i.e., triflate) or p-toluenesulfonyloxy (i.e., tosylate)), or

(b) For compounds of Formula I, wherein Q₁ has the meaning of —O—, or

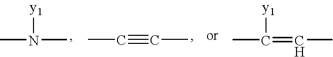

a reaction of alcohols of Formula IVb:

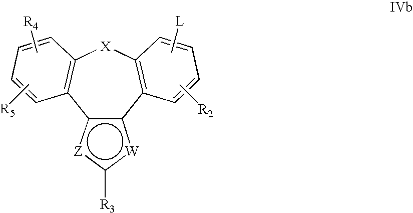

wherein L represents hydrogen, —Sn(R⁹)₃, —B(OR¹⁰)₂, or a leaving group (as defined in step (a)),
wherein R⁹ is independently chosen from hydrogen, alkyl, or alkoxy, and;
R¹⁰ is independently chosen from hydrogen or alkyl, or both R¹⁰ substituents form the cyclic structure:

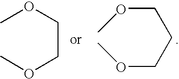

with compounds of Formula Vb,

Q₃-(CH₂)n-Q₂-(CH₂)p-A Vb

wherein Q₃ represents

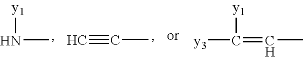

wherein Y₃ represents hydrogen, —SnR⁹, B(OR¹⁰)₂ or a leaving group (as defined for L in step (a)),

with a proviso that when L is —SnR⁹, Y₃ is not —SnR⁹, and
with an additional proviso that when L is B(OR¹⁰)₂, Y₃ is not B(OR¹⁰)₂, or

(c) For compounds of Formula I, wherein Q₁ has the meaning of —O—, —S—,

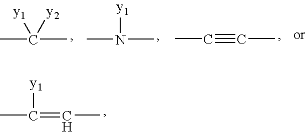

a reaction of the compounds of Formula IVc:

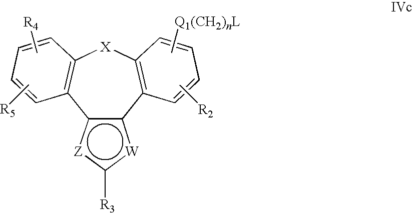

wherein
L is a leaving group as defined above;
with compounds of Formula Vc,

Q₄-(CH₂)ₚ-A Vc

wherein Q₄ represents —OH, —SH,

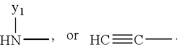

(d) The compounds of Formula I can also be synthesized through methods wherein the identity of R₁ as defined for Formula I has been established prior to the cyclization steps that follow.

### Preparation Methods:

The compounds of Formula I can be prepared by the methods disclosed above using standard protocols known in the art of organic chemistry, for example

a) Respecting the compounds of Formula I wherein synthesis occurs via the compounds of Formulae IVa and Va, reaction can occur when the group B is deprotonated upon treatment with a base and the resultant anion is reacted with Va to displace leaving group L, which may be a halogen atom (most frequently bromine, iodine or chlorine) or sulfonyloxy group (most frequently trifluoromethylsulfonyloxy or p-toluenesulfonyloxy). The condensation reaction may be carried out according to methods disclosed for the preparation of analogous compounds (Menozzi G et al., J. Heterocyclic Chem., 1997, 34:963-968 or WO 01/87890). The reaction is carried out at a temperature from 20° C. to 100° C. during 1 to 24 hours in a two-phase system (preferably with 50% NaOH/toluene) in the presence of a phase transfer catalyst (preferably benzyl triethyl ammonium chloride, benzyl triethyl ammonium bromide, cetyl trimethyl bromide). After the treatment of the reaction mixture, the products formed are isolated by recrystallization or chromatography on a silica gel column.

b) Respecting the compounds of Formula I wherein synthesis occurs via the compounds of Formulae IVb and Vb, coupling can occur in the presence of a transition metal catalyst system (e.g., Pd or Cu) through the disclosed methods, and any other methods known to those skilled in the art of sp²-sp, sp²-sp², and sp²-sp³ cross-coupling reactions. Examples include but are not limited to the Sonagashira reaction with a copper catalyst (when Q₃ is a carbon-carbon triple bond), and the Suzuki, Stille, and Kumada coupling reactions with Palladium or Nickel catalysts (when Q₃ represents a carbon-carbon double bond). Specifically, for example, a Stille cross-coupling reaction to connect R₁ to the aromatic ring of the benzodiazulene structure is carried out with the appropriate aryl halide and vinyltrialkyltin in the presence of a catalytic amount of Pd₂(dba)₃ or Pd(OAc)₂ (preferably 1-5 mol %) and an appropriate ligand (e.g., BINAP, dppe) to facilitate oxidative addition and reductive elimination during the catalytic cycle. The Stille reaction is preferably carried out in an appropriate solvent (e.g., toluene, THF) at elevated temperatures (50-110° C.) over a period of 1-24 hours.

c) Respecting the compounds of Formula I wherein synthesis occurs via the compounds of Formulae IVc and Vc, reaction can occur similar to that described for Formulae IVa and Va above. In this case the group Q₄ is deprotonated upon treatment with a base and the resultant anion is reacted with IVc to displace leaving group L. The condensation reaction may be carried out according to methods disclosed for the preparation of analogous compounds (Menozzi G et al., J. Heterocyclic Chem., 1997, 34:963-968 or WO 01/87890) as described above. Alternatively, the reaction can be performed so that the leaving group L and nucleophilic portion Q₄ are exchanged as in Formulae IVd and Vd below.

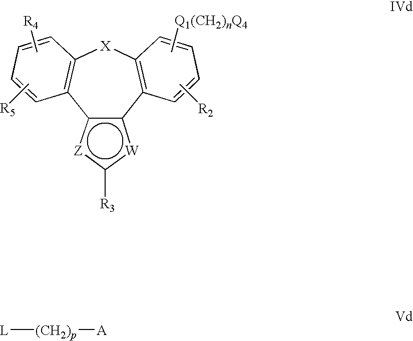

The synthesis of the compounds of Formula IV is carried out by the methods disclosed for the preparation of analogous dibenzoazulene precursors. For example, the methods disclosed in applications WO 01/87890; WO 03/097649; WO 03/097648; WO 03/099823; WO 03/099827; WO 03/084964 and WO 03/084961. For example:

d) for preparation of the compounds of Formula IV wherein W represents —O— and Z represents —O—, —NH—, —S—, or an aromatic CH, a cyclization of a compound of Formula VIa

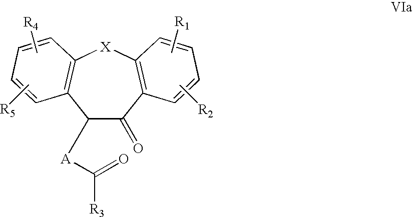

wherein
A has the meaning of —O—, —NH—, —S— or —CH₂; and
R₁ has the meaning of has the meaning defined for the compounds of Formula (I) or represents B, L, -Q₁(CH)₂L, or -Q₁(CH)₂Q₄ as defined for Formulae IVa-IVd

The cyclization of the compounds of Formula VIa is carried out by methods disclosed for the preparation of analogous compounds. Thus, e.g., compounds of Formula VIa, wherein A has the meaning of —NH—, may be cyclized by a reaction with POCl₃ in organic solvents (preferably benzene or toluene) at boiling temperature during 1 to 5 hours (Lombardino J G, J Heterocycl. Chem., 1974, 11: 17-21), whereas a cyclization of compounds of Formula VIa, wherein A has a meaning of —O—, is carried out in the presence of ammonium acetate in acetic acid at boiling temperature during 5 to 10 hours. When A has the meaning of —CH₂— cyclization is carried out in toluene or benzene at boiling temperature in 1 to 5 hours in the presence of a catalytic amount of p-toluenesulfonic acid as described in WO 03/097649. The obtained tetracyclic products may be isolated by chromatography on a silica gel column or by recrystallization from an appropriate solvent.

The starting substances for the preparation of the compounds of Formula VIa are ketones of Formula VIIIa,

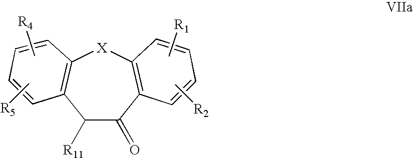

wherein R¹¹ has the meaning of H, are already known or are prepared by methods disclosed for the preparation of analogous compounds (See WO 03/084964 and WO 03/097649). By reacting sodium nitrite in ethanolic hydrochloric acid with the ketone of Formula VIIa, wherein R¹¹ is an H, the corresponding oxime is formed, which by the reduction with a metal such as zinc in acetic acid gives an amino compound of Formula VIIa, wherein R¹¹ is an NH₂ group. A similar reaction course is disclosed in U.S. Pat. No. 4,191,421. By the action of formic acid (Romo D et al., J. Am. Chem. Soc., 1998, 120: 12237-12254) or acid chlorides according to the common protocol, the compounds of Formula Va, wherein A has the meaning of —NH— group, are formed. By the acyloxylation of a corresponding ketone of Formula VIIa, wherein R¹¹ has the meaning of H atom, with Pb(OAc)₄ (Cavill G W K, Organic Oxidation Processes; 1955, 4: 4426-4429), the compounds of Formula VIa, wherein A has the meaning of —O—, are obtained. The starting reagents for the preparation of the compounds of Formula VIa wherein A represents —CH₂— are the compounds of Formula VIIa wherein R¹¹ represents hydrogen and the compounds of Formula VIIIa:

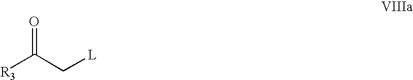

wherein L has the meaning of a leaving group ad defined above, (most frequently bromine, iodine or chlorine). The reagents VIIa and VIIIa are already known or are prepared according to methods disclosed for the preparation of analogous compounds (See, e.g., WO 03/097649).

The compounds of Formula VIa may be prepared in the presence of a strong base such as alkali hydrides (sodium hydride) or alkali amides (sodium amide) in a solvent such as dimethylformamide, dimethylsulfoxide or tetrahydrofuran at room temperature during 2 to 5 hours. The products may be isolated and purified by chromatography on a column, or may be, by means of cyclization, transferred into a corresponding furan derivative without isolation. A similar chemical sequence has already been described before (Iyer R N et al., Indian J. Chem. 1973, 11:1260-1262).

e) The starting substances for preparation of the compounds of Formula IV wherein W represents —N— and Z represents, —N—, correspond to the dibenzo-azulenes of Formula IVe:

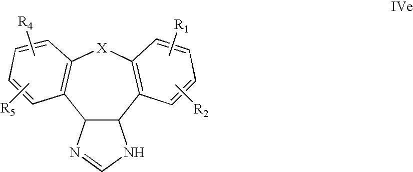

are already known or are prepared by methods disclosed for the preparation of analogous compounds (See: WO 03/099823).

Thus, e.g. compounds of Formula IVe may be prepared starting from α-diketone dibenzo-oxepine or dibenzo-thiepine. By the action of aldehyde and ammonium acetate to a-diketone, the cyclization and formation of condensed imidazole ring occur. By the reaction of paraformaldehyde a unsubstituted imidazole ring is formed. A similar reaction course is already disclosed in literature (Lombardino J G et al., J. Heterocyclic Chem., 1974, 11: 17-21). By the protection of free NH-group (WO 98/47892) of compounds of Formula IVe by the action of compounds of Formula VIIIb:

R₃-L VIIb

wherein L has the meaning of leaving group such as halogen (most frequently chlorine or bromine), the compounds IV as a mixture of 1- and 3-substituted isomers are formed. The reaction is carried out in organic solvents such as dimethylsulfoxide, tetrahydrofuran, benzene or toluene under the addition of a strong base such as sodium hydride at an increased temperature from 50° C. to 150° C. during 1 to 5 hours. The crude product may be isolated and purified by recrystallization or chromatography on a silica gel column.

f) The starting substances for preparation of the compounds of Formula IV wherein W represents —S— and Z represents, an aromatic —CH, are known for analogous compounds (See: WO 03/084961) and correspond to compounds of Formula VIIb.

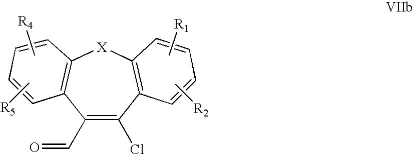

for compounds of Formula IV, wherein R₃ is alkyloxycarbonyl, a cyclization of the compound of Formula VIIb with esters of mercaptoacetic acid is appropriate. Cyclization of the compounds of Formula IV with ethyl mercaptoacetate is carried out by methods disclosed for the preparation of analogous compounds. The reaction is carried out in the presence of organic bases (preferably pyridine) at the boiling point during 1 to 5 hours. The obtained tetracyclic products may be isolated by column chromatography or by recrystallization from an appropriate solvent.

The starting substances for the preparation of the compounds of Formula VIIb, are known for analogous compounds (See: WO 03/084961) and correspond to ketones of Formula VIIa wherein R₁₁ represents hydrogen are already known or are prepared by methods disclosed for the preparation of analogous compounds. Thus, e.g., the compounds of Formula VIIa may be obtained starting from compounds of Formula IX:

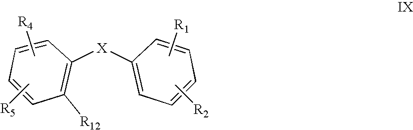

wherein R₁₂ has a meaning of a CO₂H group, in such a way that by suitable chemical transformations there is obtained a compound of Formula IX wherein R₁₂ has the meaning of CH₂CO₂H. By the action of polyphosphoric, acid, cyclization and formation of a ketone of Formula VIIa occur. A similar reaction sequence has previously been disclosed in Protiva M et al. (CS 163583, Collect. Czech. Chem. Commun., 1975, 40:1960-1965 and Collect. Czech. Chem. Commun., 1974, 39:3147-3152). Alternatively, the compound of Formula IX, wherein R₁₂ has the meaning of CH₂CO₂H, may be prepared by reacting the compound of Formula IX, wherein R₁₂ is COCH₃, with sulfur and morpholine and by hydrolyzing thioamide thus obtained (Ueda I et al., Chem. Pharm. Bull., 1975, 23:2223-2231). By the action of Vilsmeier-Haack reagent upon the corresponding ketones of Formula VIIIa, compounds of Formula VIIb (Tsuji K et al., Chem. Pharm. Bull., 1998, 46:279-286) are prepared.

Additional methods for the preparation of compounds of Formula IV wherein W represents —S— and Z represents, an aromatic —CH can be found on US Application 2003/0153750.

g) Besides the above-mentioned reactions, the compounds of Formula I may be prepared by transforming other compounds of Formula I and it is to be understood that the present invention also comprises such compounds and processes. A special example of a change of a functional group is the reduction of a compound of Formula I wherein R₁ contains an alkene or alkyne group (derived from Q₁ or Q₂ of Formula II). This reaction can occur through traditional techniques known in the art such as catalytic hydrogenation with a palladium or Platinum catalyst. Another example is the reaction of the aldehyde group with chosen phosphorous ylides resulting in a prolongation of the chain and the formation of an alkenyl substituent with carbonyl or ester groups as disclosed in HR patent application No. 20000310. These reactions are carried out in solvents such as benzene, toluene or hexane at elevated temperature (most frequently at boiling temperature).

By reacting the compounds of Formula IVc, for example, with substituted 1-alkynes in an alkaline medium (such as sodium amide in ammonia) the compounds of Formula I, wherein Q₂ is —C≡C—, are obtained. The reaction conditions of this process are disclosed in the literature. At similar reaction conditions (nucleophilic substitution) various ether, thioether or amine derivatives may be prepared.

The formylation of the compounds of Formula I by processes such as e.g. Vilsmeier acylation or reaction of n-BuLi and N,N-dimethylformamide is a further general example of a transformation. The reaction conditions of these processes are well-known in the literature.

By hydrolysis of the compounds of Formula I having nitrile, amide or ester groups, there may be prepared compounds with a carboxyl group, which are suitable intermediates for the preparation of other compounds with novel functional groups such as e.g. esters, amides, halides, anhydrides, alcohols or amines.

Oxidation or reduction reactions are a further possibility of the change of substituents in the compounds of Formula I. Most frequently used oxidation agents are peroxides (hydrogen peroxide, m-chloroperbenzoic acid or benzoyl peroxide) or permanganate, chromate or perchlorate ions. Thus e.g. by the oxidation of an alcohol group by pyridinyl dichromate or pyridinyl chlorochromate, an aldehyde group is formed, which group may be converted to a carboxyl group by further oxidation. By oxidation of the compounds of Formula I, wherein R¹ has the meaning of alkyl, with lead tetraacetate in acetic acid or with N-bromosuccinimide using a catalytic amount of benzoyl peroxide, a corresponding carbonyl derivative is obtained.

By a selective oxidation of alkylthio group, allylsulfinyl or alkylsulfonyl groups may be prepared.

By the reduction of the compounds with a nitro group, the preparation of amino compounds is made possible. The reaction is carried out under usual conditions of catalytic hydrogenation or electrochemically. By catalytic hydrogenation using palladium on carbon, alkenyl substituents may be converted to allyl ones or nitrile group can be converted to aminoalkyl.

Various substituents of the aromatic structure in the compounds of Formula I may be introduced by standard substitution reactions or by usual changes of individual functional groups. Examples of such reactions are aromatic substitutions, alkylations, halogenation, hydroxylation as well as oxidation or reduction of substituents. Reagents and reaction conditions are known from the literature. Thus e.g. by aromatic substitution a nitro group is introduced in the presence of concentrated nitric acid and sulfuric acid. By using acyl halides or alkyl halides, the introduction of an acyl group or an alkyl group is made possible. The reaction is carried out in the presence of Lewis acids such as aluminum- or iron-trichloride in conditions of Friedel-Craft reaction. By the reduction of the nitro group, an amino group is obtained, which by a diazotization reaction is converted to a suitable starting group, which may be replaced with one of the following groups: H, CN, OH, Halogen.

### Preparation Examples

The present invention is illustrated by the following Examples, which in no way represent a limitation thereof.

### Intermediate 1: [2-(4-chloro-2-methoxyphenoxy)phenyl]acetic acid

A reaction mixture of 4-chloro-2-methoxyphenol (6.31 mmol), (2-chlorophenyl)acetic acid (6.31 mmol), cooper (I) chloride (0.63 mmol), potassium carbonate (9.47 mmol) and xylene (10.0 mL) was heated under reflux for 7 hours, and then was cooled to room temperature, diluted with water and extracted with ethyl acetate. The organic extract was washed with an aqueous potassium carbonate solution. Water extracts were acidified with conc. hydrochloric acid, and then extracted with ethyl acetate. The organic extract was washed with an aqueous sodium chloride solution, dried over anhydrous Na₂SO₄ and evaporated under reduce pressure to yield the title compound as a solid product. ¹H NMR (ppm, CDCl₃): 12.37 (bs, 1H), 7.46-6.58 (m, 7H), 3.78 (s, 3H), 3.63 (s, 2H).

### Intermediate 2: 8-Chloro-6-methoxy-11H-dibenzo[b,f]oxepin-10-one

Intermediate 1 (0.05 mole) was added to polyphosphoric acid (90.0 g) at 120° C. Reaction mixture was stirred for 1 hour at 120° C. The heated reaction mixture was poured into a mixture of ice and ethyl acetate, stirred and extracted. The organic extract was washed with an aqueous sodium chloride solution, dried over anhydrous Na₂SO₄ and evaporated under reduce pressure to yield an oily product. The resulting oily product was crystallized from ethyl acetate. ¹H NMR (ppm, DMSO-d₆): 7.48-7.25 (m, 6H), 4.16 (s, 2H), 3.98 (s, 3H). MS (ES, m/z): 274.98 [M+H]⁺, calculated 275.05.

### Intermediate 3: 2,11-Dichloro-4-methoxy-dibenzo[b,f]oxepine-10-carbaldehyde

Phosphorus oxychloride (10.9 mmol) was slowly added to a mixture of N,N′-dimethylformamide (16.4 mmol) and dichloromethane (1.85 mL) at 0° C. The reaction mixture was stirred 30 minutes at room temperature. A solution of Intermediate 2 (3.64 mmole) in dichloromethane (2.80 mL) was added to reaction mixture. The reaction mixture was stirred for 1 hour at room temperature, and then for 1 hour at 48° C. The mixture was then cooled at room temperature. An aqueous solution of sodium acetate was slowly added to reaction mixture. The reaction mixture was extracted with dichloromethane. The organic extract was washed with an aqueous sodium chloride solution, dried over anhydrous Na₂SO₄ and evaporated under reduce pressure to yield a solid product. ¹H NMR (ppm, DMSO-d₆): 10.52 (s, 1H), 7.96-7.25 (m, 6H), 3.95 (s, 3H). MS (ES, m/z): 320.92 [M+H]⁺, calculated 321.01.

### Intermediate 4: 11-Chloro-9-methoxy-8-oxa-1-thia-dibenzo[e,h]azulene-2-carboxylic acid ethyl ester

Ethyl 2-mercaptoacetate (3.49 mmol) and triethylamine (8.72 mmol) were added to a solution of Intermediate 3 in pyridine (7.0 mL). The reaction mixture was stirred 1 hour at 80° C. and then cooled to room temperature and evaporated under reduce pressure. After evaporation, water was added to the residue and then it was extracted with dichloromethane. The organic extract was washed with an aqueous hydrochloric acid and sodium chloride solutions, dried over anhydrous Na₂SO₄ and evaporated under reduced pressure to yield a solid product. The resulting solid product was crystallized from ethyl acetate. ¹H NMR (ppm, DMSO-d₆): 8.12 (s, 1H), 7.71-7.12 (m, 6H), 4.37 (q, 2H), 3.95 (s, 3H), 1.35 (t, 3H). MS (ES, m/z): 386.95 [M+H]⁺, calculated 387.05.

### Intermediate 5: 11-Chloro-9-hydroxy-8-oxa-1-thia-dibenzo[e,h]azulene-2-carboxylic acid ethyl ester

A 1.0 M solution of boron tribromide (7.77 mmol) was slowly dropped to a solution of Intermediate 4 (1.29 mmol) in dichloromethane (20.0 mL). The reaction mixture was stirred 4 hours at room temperature, diluted with water and extracted with dichloromethane. The organic extract was washed with an aqueous hydrochloric acid and sodium chloride solutions, dried over anhydrous Na₂SO₄ and evaporated under reduce pressure. After purification of the evaporated residue by chromatography on a silica gel column, a solid product was isolated. ¹H NMR (ppm, DMSO-d₆): 10.50 (s, 1H), 8.17 (s, 1H), 8.10-7.01 (m, 6H), 4.37 (q, 2H), 1.35 (t, 3H). MS (ES, m/z): 371.00 [M−H]⁻, calculated 371.02.

### Example 1

### 11-Chloro-9-(3-dimethylaminopropoxy)-8-oxa-1-thia-dibenzo[e,h]azulene-2-carboxylic acid ethyl ester

Potassium carbonate (0.56 mmole) was added to a solution of Intermediate 5 (0.22 mmol) in 1N,N′-dimethylformamide (3.0 mL). The reaction mixture was heated to 100° C. and 3-dimethylaminopropylchloride hydrochloride (0.27 mmol) was added. The reaction mixture was stirred for 4 hours at 100° C., and then cooled to room temperature and evaporated under reduce pressure. After evaporation, water was added to the residue and the resultant mixture was extracted with ethyl acetate. The organic extract was washed with aqueous hydrochloric acid and sodium chloride solutions, dried over anhydrous Na₂SO₄ and evaporated under reduce pressure. After purification of the evaporated residue by chromatography on a silica gel column, a solid product was isolated. ¹H NMR (ppm, CDCl₃): 8.03 (s, 1H), 7.53-6.98 (m, 6H), 4.41 (q, 2H), 4.14 (t, 2H), 2.72 (t, 2H), 2.42 (s, 6H), 2.20 (qn, 2H), 1.42 (t, 3H). MS (ES, m/z): 458.02 [M+H]⁺, calculated 458.12.

### Example 2

### 11-Chloro-9-(3-dimethylamino-propoxy)-8-oxa-1-thia-dibenzo[e,h]azulene-2-carboxylic acid ethyl ester citrate salt

To a stirred ethanol solution of Example 1 (0.25 g, 0.546 mmol in 5 mL of ethanol), previously cooled at 0° C., an ethanol solution of citric acid was dropped (0.1146 g, 0.546 mmol in 5 mL of ethanol). When addition of citric acid was completed, ethanol was evaporated to yield a light yellow solid product (0.27 g); MS (m/z, ES+): 458 [MH]+; m.p. 80-87° C.; pKa 6.90; logP 4.5; solubility (S) 167 mg/mL (at 25° C. and ionic strength 0.15 M).

### Example 3

### 2-Hydroxymethyl-1′-Chloro-9-(3-diethylaminopropoxy)-8-oxa-1-thia-dibenzo[e,h]azulene

To a suspension of LiAlH₄ (0.29 g, 7.21 mmol) in dry Et₂O (15.0 mL) a suspension of 11-chloro-9-(3-dimethylaminopropoxy)-8-oxa-1-thia-dibenzo[e,h]azulene-2-carboxylic acid ethyl ester (2.20 g, 4.80 mmol) in dry Et₂O (15.0 mL) was added dropwise. The reaction mixture was stirred for 3 hours at room temperature. To the reaction mixture, water was added dropwise. After the precipitate was formed, reaction mixture was filtered and precipitate washed with Et₂O (25.0 mL) and EtOAc (25.0 mL). Mother liquor was dried over anhydrous Na₂SO₄ and evaporated in vacuum yielding yellowish crude product (2.0 g). Crude product was recrystallized from EtOAc (5 mL). Yield: (1.75 g, 87.4%). ¹H NMR (300 MHz, DMSO-d₆) δ/ppm: 7.61-7.20 (m, 6H), 7.01 (dd, J=3.0 Hz, 1H), 4.74 (s, 2H), 4.14 (t, J=6.2 Hz, 2H), 2.46 (t, J=7.0 Hz, 2H), 2.18 (s, 6H), 1.97 (p, J=5.5 Hz, 2H). MS (ES, m/z): 415.9 ([M+H]+, calculated 416.2).

### Example 4

### 11-Chloro-9-(3-dimethylaminopropoxy)-8-oxa-1-thia-dibenzo[e,h]azulene-2-ylmethyl acetate

To a solution of 2-hydroxymethyl-11-Chloro-9-(3-diethylaminopropoxy)-8-oxa-1-thia-dibenzo[e,h]azulene (100.0 mg, 0.24 mmol) in pyridine (3.0 mL) at 0° C., Ac₂O (227.3 μL, 2.4 mmol) and DMAP (3.0 mg, 0.02 mmol) were added. The reaction mixture was stirred for 5 hours at room temperature. The reaction mixture was poured over ice and extracted with EtOAc (3×10 mL). The combined organic extracts were washed with brine (15 mL), dried over anhydrous Na₂SO₄ and evaporated in vacuum yielding brown crude product (110 mg). Crude compound was purified on preparative LC-MS. After purification 57.1 mg (52.0%) of crude product was obtained ¹H NMR (300 MHz, CDCl₃) δ/ppm: 7.48-7.45 (m, 1H), 7.37-7.34 (m, 2H), 7.24-7.19 (m, 2H), 7.02 (dd, J=2.3 Hz, 1H), 6.92 (dd, J=2.3 Hz, 1H), 5.30 (s, 2H), 4.14 (t, J=6.1 Hz, 2H), 2.84 (t, J=7.1 Hz, 2H), 2.50 (s, 6H), 2.26 (p, J=7.9 Hz, 2H), 2.14 (s, 3H). MS (ES, m/z): 458.0 ([M+H]⁺, calcd. 458.1).

### General Procedure for Amide Derivative Formation by Formation of Active Ester Bounded to the Resin

To a 4-hydroxy-2,3,5,6-tetrafluorobenzamidomethyl polystyrene resin (PS-TFP, loading: 1.23 mmol/g) (57.5 mg, 0.07 mmol), a solution of 11-chloro-9-(3-dimethylaminopropoxy)-8-oxa-1-thia-dibenzo[e,h]azulene-2-carboxylic acid (46 mg, 0.14 mmol) in dry DMF (dried over molecular sieves) (1.0 mL) was added followed by a DMAP (5.13 mg, 0.042 mmol) solution in dry DMF (0.53 mL). The mixture was shaken for 10 minutes, and a solution of DIC (47.7 μL, 0.31 mmol) in dry DMF (0.18 mL) was added. The reaction mixture was shaken for 16 hours at room temperature. The mixture was filtered and the resin was washed with DMF (3×1.5 mL), THF (3×1.5 mL), DMF (3×1.5 mL) and CH₂Cl₂ (3×1.5 mL) and dried. This procedure produces activated ester bounded to the resin which serves as intermediates to amide synthesis.

### Amide Synthesis Procedure

To a suspension of PS-TFP active ester resin (0.07 mmol) in dry DMF (dried over molecular sieves) (1.0 mL) a suspension of amine (0.07 mmol) in dry DMF (1.0 mL) was added, followed by a DIPEA (13.6 μL, 0.08 mmol) solution in dry DMF (0.5 mL). The reaction mixture was shaken 3 hours at room temperature. The solution was filtered and the resin was washed with DMF (4×1.5 mL). The combined mother liquor were concentrated affording the desired amide

### Example 5

### 11-Chloro-9-(3-dimethylaminopropoxy)-8-oxa-1-thia-dibenzo[e,h]azulene-2-carboxylic acid amide

The title compound was prepared according to previously described general procedure affording of 29 mg of crude product which was purified on preparative LC-MS. After purification 11.7 mg (38.4%) of white powder was obtained. MS (ES, m/z) ([M+H]⁺): 430.0

### Example 6

### N,N-Dimethyl-11-chloro-9-(3-dimethylaminopropoxy)-8-oxa-1-thia-dibenzo[e,h]azulene-2-carboxamide

The title compound was prepared according to previously described general procedure affording 43% of crude product which was purified on preparative LC-MS. After purification 25% of pure product was obtained as white powder. MS (ES, m/z) ([M+H]⁺): 458.0

### Example 7

### N,N-Diethyl-11-chloro-9-(3-dimethylaminopropoxy)-8-oxa-1-thia-dibenzo[e,h]azulene-2-carboxamide

The title compound was prepared according to previously described general procedure affording 39% of crude product which was purified on preparative LC-MS. After purification 31% of white powder was obtained. MS (ES, m/z) ([M+H]⁺): 486.0

### Biological Testing

The activity of compounds of the present invention to inhibit monoamine reuptake was determined in vitro and in vivo by the following experiments:

### In Vitro Experiments

Methods employed in this study have been adapted from the scientific literature to maximize reliability and reproducibility. Reference standards were run as an integral part of each assay to ensure the validity of the results obtained. Assays were performed under the following conditions:

Serotonin Transporter (SERT): (See Gu H, Wall S and Rudnick G., Stable expression of biogenic amine transporters reveals differences in inhibitor sensitivity, kinetics, and ion dependence., J. Biol. Chem. 1994, 269(10):7124-7130).

| | |
|---|---|
| Source: | Human recombinant HEK-293 cells |
| Ligand: | 0.15 nM [¹²⁵I] RTI-55 ((3 beta- |
| | (4-iodophenyl)tropan-2 beta-carboxylic acid |
| | methyl ester; inhibitor) |
| Vehicle: | 1% DMSO |
| Incubation Time/Temp: | 3 hours at 4° C. |
| Incubation Buffer: | 1 μM Leupeptin, 100 mM NaCl, 10 μM |
| | PMSF, 50 mM Tris-HCl, pH 7.4 |
| Non-Specific Ligand: | 10 μM Imipramine |
| K_{D1}: | 0.17 nM |
| K_{D2}: | 2.2 μM |
| B_{MAX1}: | 0.41 pmole/mg Protein |
| B_{MAX2}: | 790 pmole/mg |
| Specific Binding: | 95% |
| Quantitation Method: | Radioligand Binding |
| Significance Criteria: | >50% of max stimulation or inhibition |

Dopamine Transporter (DAT): (See Gu H, Wall S and Rudnick G., Stable expression of biogenic amine transporters reveals differences in inhibitor sensitivity, kinetics, and ion dependence., J. Biol. Chem. 1994, 269(10):7124-7130).

| | |
|---|---|
| Source: | Human recombinant CHO cells |
| Ligand: | 0.15 nM [¹²⁵I] RTI-55 |
| Vehicle: | 1% DMSO |
| Incubation Time/Temp: | 3 hours at 4° C. |
| Incubation Buffer: | 1 μM Leupeptin, 100 mM NaCl, 10 μM PMSF, |
| | 50 mM Tris-HCl, pH 7.4 |
| Non-Specific Ligand: | 10 μM Nomifensine |
| K_{D}: | 0.58 νM |
| B_{MAX}: | 0.047 pmole/mg Protein |
| Specific Binding: | 90% |
| Quantitation Method: | Radioligand Binding |
| Significance Criteria: | >50% of max stimulation or inhibition |

Norepinephrine Transporter (NET): (See Galli A., De Felice L., Duke B-J., Moore K. and Blakely R., Sodium dependent norepinephrine induced currents in norepinephrine transporter transfected HEK293 cells blocked by cocaine and antidepressants., J Exp Biol. 1995, 198:2197-2212).

| | |
|---|---|
| Source: | Human recombinant MDCK cells |
| Ligand: | 0.2 nM [¹²⁵I] RTI-55 |
| Vehicle: | 1% DMSO |
| Incubation Time/Temp: | 3 hours at 4° C. |
| Incubation Buffer: | 1 μM Leupeptin, 100 mM NaCl, 10 μM PMSF, |
| | 50 mM Tris-HCl, pH 7.4 |
| Non-specific Ligand: | 10 μM Desipramine |
| K_{D}: | 0.024 μM |
| B_{MAX}: | 2.5 pmole/mg Protein |
| Specific Binding: | 75% |
| Quantitation Method: | Radioligand Binding |
| Significance Criteria: | >50% of max stimulation or inhibition |

Results are presented as IC₅₀ values, which were determined by a non-linear, least squares regression analysis; IC₅₀=[1+L(1)/K_{D})/(1+L(2)/K_{D})]×IC₅₀(2), where IC₅₀(2) is a known value for the K_{D} of the ligand) and inhibition constants Ki (Kᵢ=IC₅₀/(1+L/K_{D}), which were calculated using the equation of Cheng and Plusoff (Biochem. Pharmacol. 22:3099-3108, 1973) using the observed IC₅₀ of the tested compound, the concentration of radioligand employed in the assay, and the historical values for the K_{D} of the ligand.

Compounds demonstrating IC₅₀ and Kᵢ at concentrations lower than 1 μM were considered to be active. The compounds described in examples 1-7 exhibited binding affinity to serotonin, dopamine and norepinephrine transporters, expressed as IC₅₀ and Kᵢ values less than 1 μM and were considered to be active.

It is anticipated that similar results will be observed for other compounds described by Formula I which is subject of the invention.

### In Vivo Experiments

### Tail-Suspension Test in Mice: Evaluation of Antidepressive Efficacy

The aim of this study was to evaluate potential antidepressant activity of selected compounds Formula I. The tail suspension test has been routinely employed to test potential antidepressant compounds (See H. G. Vogel and W. H. Vogel, Drug Discovery and Evaluation, Pharmacological Assays, Springer-Verlag, 1997, p 304-305). The percentage of drug-treated animals showing the passive behavior (i.e., the immobility that mice display after active and unsuccessful attempts to escape when suspended by the tail) is measured and compared with vehicle-treated control animals. The duration of immobility is recorded for about 5 minutes. Mice are considered immobile when they hang passively and completely motionless for at least 1 minute. Active antidepressants reduce the immobility.

### Methods

Groups of 9 animals were treated with compounds encompassed by Formula I (0.1-10 mg/kg; s.c.), or fluoxetine (10 mg/kg i.p.), and one group of 15 animals was treated with the vehicle by s.c. injection as a positive control. All animals were treated for 30 min prior to testing. The percentage of drug-treated animals showing passive behavior was determined and compared with vehicle-treated controls.

### Results

In this experiment, 70% of vehicle-treated control animals exhibited passive behavior (i.e., increased immobility). In the group treated with standard fluoxetine, 22% of animals were depressive (i.e., exhibited passive behavior). In a group treated with the compounds of Formula I described in examples 1-7, 12-44% of the animals were depressive.

In this experiment active compounds are compounds which after s.c. administration in doses of 0.1-10 mg/kg to mice, produced a decrease in passive behavior, i.e., reduced immobility by at least 40% compared to vehicle-treated control animals.

It is anticipated that similar results will be observed for other compounds described by Formula I which is subject of the invention.

### Determination of Analgesic Activity

Writhing Test

In this assay, pain is induced by the injection of an irritant, most frequently acetic acid, into the peritoneal cavity of mice. Animals react with characteristic writhings (Collier H O J et al., Pharmac. Chemother., 1968, 32:295-310; Fukawa K et al., J. Pharmacol. Meth., 1980, 4:251-259; Schweizer A et al., Agents Actions, 1988, 23:29-31). This assay is a standard model for the determination of analgesic activity of compounds (see H. G. Vogel and W. H. Vogel, Drug Discovery and Evaluation, Pharmacological Assays, Springer-Verlag, 1997, p 382-384).

### Methods

Briefly: male Balb/C mice (Charles River, Italy), age 8 to 12 weeks, are used. The control group receives 0.2 ml of a 1% methyl cellulose suspension p.o. 30 minutes prior to i.p. application of acetic acid in a concentration of 0.6%, whereas test groups receive standard analgesic (acetylsalicylic acid), or test compounds of Formula I at various concentrations in 1% methyl cellulose p.o. 30 minutes prior to i.p. application of 0.6% acetic acid (volume 0.1 ml/10 g). The mice are placed individually under glass funnels and the number of writhings is registered for a duration of 20 minutes for each animal (following a 5 minute initial period). A writhe is indicated by stretching of the abdomen with simultaneous stretching of at least one hind limb. The percentage of writhing inhibition was calculated according to the following equation:

% inhibition=(mean value of number of writhings in the control group−number of writhings in the test group)/number of writhings in the control group*100.

Active compounds are those compounds showing at least as much analgesic activity as acetylsalicylic acid, or better, as determined by the decrease in the percent of writhings.

It is anticipated that analgesic activity will be observed for the compounds of the invention.

Chronic Pain Model—Formalin Test in Mice

The formalin test in rodents has been used as a chronic pain model which is sensitive to centrally active analgesic agents (See Dubuisson and Dennis, The Formalin test: A quantitative study of the analgesic effects of morphine, meperidine and brain stem scintillation in rats and cats. Pain, 1977, 4, 161-174).

### Methods

Mice are administered 10% formalin into the dorsal portion of the front paw. The test drugs are administered simultaneously either s.c. or orally at 0.1 mg kg to 10 mg/kg. As a standard (positive control) morphine is administered s.c. at an effective dose of 1.7 mg/kg. Negative controls are animals not treated with formalin. Each individual animal is then placed into a clear plastic cage for observation. Pain responses are indicated by elevation or favoring of the treated paw, or excessive licking and/or biting of the treated paw. Readings are taken at 30 and 60 minutes and scored according to a pain scale. Analgesic response or protection is indicated if both paws are resting on the floor with no obvious favoring of the injected paw.

### Evaluation of Results

ED₅₀ values for protection are calculated for each compound at various doses.

Compounds demonstrating analgesic effects of ED₅₀ or better compared to formalin-treated animals not administered any analgesic will be designated effective.

It is anticipated that analgesic effects will be observed for the compounds of the invention.

Patents, patent applications, publications, and protocols which are cited throughout this application are incorporated herein by reference in their entireties for all purposes.

What is claimed is:

1. A compound having Formula I 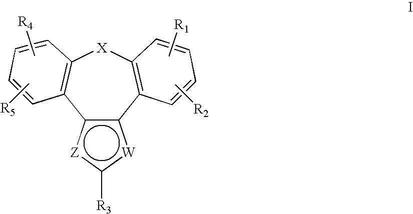
wherein,
X is selected from —CH₂—, —O—; —S—, or —NR⁶—, wherein R⁶ has meaning of hydrogen, C₁-C₄ alkyl, C₇-C₁₀ arylalkyl, C₂-C₅ alkanoyl, C₇-C₁₀ aryloyl, or C₂-C₇ alkyloxycarbonyl;
W and Z independently from each other have the meaning of oxygen, sulfur, an aromatic CH, or NR⁷ wherein R⁷ has meaning of hydrogen, C₁-C₄ alkyl, C₇-C₁₀ arylalkyl, C₂-C₅ alkanoyl, C₇-C₁₀ aryloyl, or C₂-C₇ alkyloxycarbonyl with a proviso that W and Z cannot simultaneously be oxygen, sulfur, or an aromatic CH;
R¹ denotes a substituent represented by Formula II.
Q₁-(CH₂)n-Q₂-(CH₂)p-A II
wherein Q₁ and Q₂ are independently selected from oxygen, sulfur or group selected from: 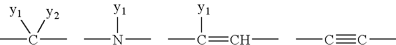
wherein Y₁ is selected from hydrogen; C₁-C₄ alkyl which is unsubstituted or is substituted by 1 to 3 substituents selected from the group consisting of halogen, hydroxy, C₁-C₄ alkoxy, C₁-C₄ alkoxycarbonyl, thiol, C₁-C₄ alkylthio, amino, N—(C₁-C₄) alkylamino, N,N-di(C₁-C₄ alkyl)-amino, sulfonyl, C₁-C₄ alkylsulfonyl, sulfinyl and C₁-C₄ alkylsulfinyl; aryl which is unsubstituted or is substituted by one or two substituents selected from the group consisting of halogen, C₁-C₄ alkyl, cyano, nitro, hydroxy, C₁-C₄ alkoxy, thiol, C₁-C₄ alkylthio, amino, N—(C₁-C₄) alkylamino, N,N-di(C₁-C₄ alkyl)-amino sulfonyl, C₁-C₄ alkylsulfonyl, sulfinyl and C₁-C₄ alkylsulfinyl; hydroxy; C₁-C₄ alkoxy; C₁-C₄ alkanoyl; sulfonyl; C₁-C₄ alkylsulfonyl; sulfinyl; C₁-C₄ alkylsulfinyl; and
Y₂ is selected from hydrogen; halogen; C₁-C₄ alkyl which is unsubstituted or is substituted by 1 to 3 substituents selected from the group consisting of halogen, hydroxy, C₁-C₄ alkoxy, C₁-C₄ alkoxycarbonyl, thiol, C₁-C₄ alkylthio, amino, N—(C₁-C₄) alkylamino, N,N-di(C₁-C₄ alkyl)-amino, sulfonyl, C₁-C₄ alkylsulfonyl, sulfinyl and C₁-C₄ alkylsulfinyl; aryl which is unsubstituted or is substituted by one or two substituents selected from the group consisting of halogen, C₁-C₄ alkyl, cyano, nitro, hydroxy, C₁-C₄ alkoxy, thiol, C₁-C₄ alkylthio, amino, N—(C₁-C₄)alkylamino, N,N-di(C₁-C₄ alkyl)-amino, sulfonyl, C₁-C₄ alkylsulfonyl, sulfinyl and C₁-C₄ alkylsulfinyl; hydroxy; C₁-C₄ alkoxy; C₁-C₄ alkanoyl; thiol; C₁-C₄ alkylthio; sulfonyl; C₁-C₄ alkylsulfonyl; sulfinyl; C₁-C₄ alkylsulfinyl; cyano; nitro;
with a proviso that at least one of substituents Y₁ and Y₂ when attached to carbon atom is hydrogen;
A is selected from amino, N—(C₁-C₇ alkyl)amino, N,N-di(C₁-C₇ alkyl)amino, aryl which is unsubstituted or is substituted by one or two substituents selected from the group consisting of halogen, C₁-C₄ alkyl, cyano, nitro, hydroxy, C₁-C₄ alkoxy, thiol, C₁-C₄ alkylthio, amino, N—(C₁-C₄)alkylamino, N,N-di(C₁-C₄-alkyl)-amino, sulfonyl, C₁-C₄ alkylsulfonyl, sulfinyl and C₁-C₄ alkylsulfinyl; heterocyclic or heteroaryl group selected from the group consisting of morpholine-4-yl, piperidine-1-yl, pyrrolidine-1-yl, imidazole-1-yl and piperazine-1-yl, or represented by the structure of Formula III: 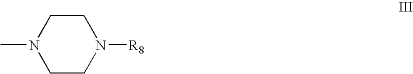
wherein R⁸ is selected from hydrogen, C₁-C₇ alkyl which is unsubstituted or is substituted by 1 to 3 substituents selected from the group consisting of halogen, hydroxy, C₁-C₄ alkoxy, C₁-C₄ alkoxycarbonyl, thiol, C₁-C₄ alkylthio, amino, N—(C₁-C₄) alkylamino, N,N-di(C₁-C₄ alkyl)-amino, sulfonyl, C₁-C₄ alkylsulfonyl, sulfinyl and C₁-C₄ alkylsulfinyl: C₁-C₇ alkenyl which is unsubstituted or is substituted by 1 to 3 halogen; C₂-C₇ alkynyl, aryl which is unsubstituted or is substituted by one or two substituents selected from the group consisting of halogen, C₁-C₄ alkyl, cyano, nitro, hydroxy, C₁-C₄ alkoxy, C₁-C₄ alkoxycarbonyl, thiol, C₁-C₄ alkylthio, amino, N—(C₁-C₄) alkylamino, N,N-di(C₁-C₄ alkyl)-amino, sulfonyl, C₁-C₄ alkylsulfonyl, sulfinyl and C₁-C₄ alkylsulfinyl; heteroaryl; heterocyclyl; C₁-C₇ alkoxy; C₁-C₇ alkylthio C₁-C₇ alkanoyl; aryloyl; oxo-C₁-C₇ alkyl; C₁-C₇ alkanoyloxy; carboxy; C₁-C₇ alkyloxycarbonyl which is unsubstituted or is substituted by 1 to 3 substituents selected from the group consisting of halogen, hydroxy, C₁-C₄ alkoxy, C₁-C₄ alkoxycarbonyl, thiol, C₁-C_{X} alkylthio, amino, N—(C₁-C₄) alkylamino. N,N-di(C₁-C₄ alkyl)-amino, sulfonyl, C₁-C₄ alkylsulfonyl, sulfinyl and C₁-C₄ alkylsulfinyl; or C₇-C₁₀ aryloxycarbonyl which is unsubstituted or is substituted by 1 to 3 substituents selected from the group consisting of halogen, hydroxy, C₁-C₄ alkoxy, C₁-C₄ alkoxycarbonyl, thiol, C₁-C₄ alkylthio, amino, N—(C₁-C₄) alkylamino, N,N-di(C₁-C₄ alkyl)-amino, sulfonyl, C₁-C₄ alkylsulfonyl, sulfinyl and C₁-C₄ alkylsulfinyl; carbamoyl: N—(C₁-C₇ alkyl)carbamoyl; N,N-di(C₁-C₇-alkyl)carbamoyl; cyano-C₁-C₇ alkyl; C₁-C₇ alkylsulfonyl; C₁-C₇ alkylsulfinyl;
n and p are each independently integers from 0 to 5 with proviso that when n has the meaning of zero, C₁ and C₂ cannot simultaneously be oxygen, sulfur or 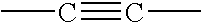
and when p has the meaning of zero, Q2 cannot be oxygen, sulfur or 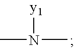
R² is halogen;
R³, R⁴ and R⁵ are independently selected from hydrogen, halo, C₁-C₇ alkyl which is unsubstituted or is substituted by 1 to 3 substituents selected from the group consisting of halogen, hydroxy C₁-C₄ alkoxy, C₁-C₄ alkoxycarbonyl, thiol, C₁-C₄ alkylthio, amino, N—(C₁-C₄) alkylamino, N,N-di(C₁-C₄ alkyl)-amino, sulfonyl, C₁-C₄ alkylsulfonyl, sulfinyl and C₁-C₄ alkylsulfinyl, hydroxy, C₁-C₇ alkoxy, thiol, C₁-C₇ alkylthio, amino, N—(C₁-C₇ alkyl)amino, N,N-di(C₁-C₇ alkyl)amino, C₁-C₇ alkanoyloxy, carboxy, C₁-C_{X} alkyloxycarbonyl which is unsubstituted or is substituted by 1 to 3 substituents selected from the group consisting of halogen, hydroxy, C₁-C₄ alkoxy (preferably methoxy or ethoxy), C₁-C₄ alkoxycarbonyl, thiol, C₁-C₄ alkylthio, amino, N—(C₁-C₄) alkylamino, N,N-di(C₁-C₄ alkyl)-amino, sulfonyl, C₁-C₄ alkylsulfonyl, sulfinyl and C₁-C₄ alkylsulfinyl, C₇-C₁₀ aryloxycarbonyl which is unsubstituted or is substituted by 1 to 3 substituents selected from the group consisting of halogen, hydroxy, C₁-C₄ alkoxy, thiol, C₁-C₄ alkoxycarbonyl, C₁-C₄ alkylthio, amino, N—(C₁-C₄) alkylamino, N,N-di(C₁-C₄ alkyl)-amino, sulfonyl, C₁-4 alkylsulfonyl, sulfinyl and C₁-C₄ alkylsulfinyl, carbamoyl, N—(C₁-C₇-alkyl)carbamoyl, N,N-di(C₁-C₇-alkyl)carbamoyl, cyano, cyano-C₁-C₇ alkyl, sulfonyl, C₁-C₇ alkylsulfonyl, sulfinyl, C₁-C₇ alkylsulfinyl or nitro group;
and pharmacologically acceptable salts or solvates thereof.

2. The compound of claim 1, wherein X represents O or S

3. The compound of claim 1 wherein R¹ is represented by Formula II:
Q₁-(CH₂)n-Q₂-(CH₂)p-A II
wherein Q₁ and Q₂ are independently selected from oxygen, sulfur or group selected from, 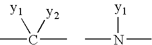
wherein Y₁ and Y₂ are independently selected from hydrogen and C₁-C₄ alkyl;
A is selected from amino, N—(C₁-C₇ alkyl)amino, N,N-di(C₁-C₇ alkyl)amino, heterocyclic or heteroaryl group selected from the group consisting of morpholine-4-yl, piperidine-1-yl, pyrrolidine-1-yl, imidazole-1-yl and piperazine-1-yl, or represented by the structure of Formula III: 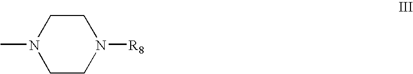
wherein R⁸ is selected from hydrogen; C₁-C₇ alkyl which is unsubstituted or is substituted as defined above; C₁-C₇ alkoxy; C₁-C₇ alkylthio; C₁-C₇ alkanoyl; aryloyl; oxo-C₁-C₇ alkyl; C₁-C₇ alkanoyloxy: carboxy; C₁-C₇ alkyloxycarbonyl which is unsubstituted or is substituted by; or C₇-C₁₀ aryloxycarbonyl which is unsubstituted or is substituted by; carbamoyl; N—(C₁-C₇alkyl)carbamoyl; N,N-di(C₁-C₇-alkyl)carbamoyl; cyano-C₁-C₇ alkyl; C₁-C₇ alkylsulfonyl; C₁-C₇ alkylsulfinyl;
n and p are each independently integers from 0 to 5 with proviso that when n has the meaning of zero, Q1 and Q2 cannot simultaneously be oxygen, sulfur or 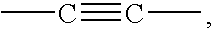
and when p has the meaning of zero, Q2 cannot be oxygen, sulfur or

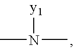

4. The compound of claim 1 wherein R⁴ and R⁵ are independently selected from hydrogen, halo, C₁-C₇ alkyl which is unsubstituted or is substituted as specified above, hydroxy, C₁-C₇ alkoxy, thiol, C₁-C₇ alkylthio and R³ is C₁-C₇ alkanoyloxy, carboxy, C₁-C₇ alkyloxycarbonyl which is unsubstituted or is substituted by, C₇-C₁₀ aryloxycarbonyl which is unsubstituted or is substituted by, carbamoyl, N—(C₁-C₇-alkyl)carbamoyl, N,N-di(C₁-C₇-alkyl)carbamoyl, cyano group; and
pharmacologically acceptable salts and solvates thereof.

5. The compound of claim 1, wherein
X is —O—
W is an aromatic —CH—
Z is —S—
R¹ is represented by Formula II:
Q₁-(CH₂)n-Q₂-(CH₂)p-A; wherein
Q₁ is oxygen;
Q₂ is 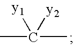
wherein
Y₁ and are hydrogen, and
A is N,N-di(C₁-C₂ alkyl)amino;
n and p are 1
R² is chlorine;
R³ is hydroxymethyl, acetylmethyl, aminocarbonyl, dimethylaminocarbonyl, diethylaminocarbonyl, or ethoxycarbonyl; and
R⁴ and R⁵ are hydrogen.

6. The compound of claim 1 wherein the compound of Formula I is a 2-Substituted-11-chloro-9-(3-dimethylaminopropoxy)-8-oxa-1-thia-dibenzo[e,h]azulene, wherein R³ represents C₁-C₇ alkyloxycarbonyl, carbamoyl, N—(C₁-C₇-alkyl)carbamoyl, N,N-di(C₁-C₇-alkyl)carbamoyl, or cyano group; and
pharmacologically acceptable salts and solvates thereof.

7. A compound selected from:
11-Chloro-9-(3-dimethylaminopropoxy)-8-oxa-1-thia-dibenzo[e,h]azulene-2-carboxylic acid ethyl ester;
2-Hydroxymethyl-11-chloro-9-(3-dimethylaminopropoxy)-8-oxa-1-thia-dibenzo[e,h]azulene;
11-Chloro-9-(3-dimethylaminopropoxy)-8-oxa-1-thia-dibenzo[e,h]azulene-2-ylmethyl acetate;
11-Chloro-9-(3-dimethylaminopropoxy)-8-oxa-1-thia-dibenzo[e,h]azulene-2-carboxamide;
N,N-Dimethyl-11-chloro-9-(3-dimethylaminopropoxy)-8-oxa-1-thia-dibenzo(e,h)azulene-2-carboxamide;
N,N-Diethyl-11-chloro-9-(3-dimethylaminopropoxy)-8-oxa-1-thia-dibenzo[e,h]azulene-2-carboxamide;
pharmaceutically acceptable salts thereof.

8. A pharmaceutical composition, comprising a therapeutically effective amount of a compound of a claim 1 or a pharmaceutically acceptable salt or solvate thereof, together with at least one pharmaceutically acceptable carrier or diluent.

9. (canceled)

10. (canceled)

11. A method of treating a disorder or disease responsive to the inhibition of monoamine neurotransmitters reuptake in the CNS, which comprises administering to a living animal body in need thereof, including a human in need thereof, a therapeutically effective amount of compound of Formula I or a pharmacologically acceptable salt or hydrate thereof.

12. The method of claim 11 wherein the disorder or disease responsive to the inhibition of monoamine neurotransmitters reuptake in the CNS is depression (e.g., depression in cancer patients, depression in Parkinson's patients, postmyocardial infarction depression, subsyndromal symptomatic depression, depression in infertile women, pediatric depression, major depression, single episode depression, recurrent depression, child abuse induced depression, and post partum depression), generalized anxiety disorder, phobias (e.g., agoraphobia, social phobia and simple phobias), post-traumatic stress syndrome, avoidant personality disorder, premature ejaculation, eating disorders (e.g., anorexia nervosa and bulimia nervosa), obesity, chemical dependencies (e.g., addictions to alcohol, cocaine, heroin, phenobarbital, nicotine and benzodiazepines), cluster headache, migraine, pain. Alzheimer's disease, obsessive compulsive disorder, panic disorder, memory disorders (e.g., dementia, amnesic disorders, and age-related cognitive decline (ARCD), Parkinson's diseases (e.g., dementia in Parkinson's disease, neuroleptics-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, attention deficit hyperactivity disorder (ADHD), chronic paroxysmal hemicrania and headache (associated with vascular disorders) is treated.
